SEQUENCE LISTING (10 sequences)

SEQ ID NO: 1 - protein, 10 aa, Artificial
  Belimumab CDRH1
GGTFNNNAIN

SEQ ID NO: 2 - protein, 17 aa, Artificial
  Belimumab CDRH2
GIIPMFGTAKYSQNFQG

SEQ ID NO: 3 - protein, 14 aa, Artificial
  Belimumab CDRH3
SRDLLLFPHHALSP

SEQ ID NO: 4 - protein, 11 aa, Artificial
  Belimumab CDRL1
QGDSLRSYYAS

SEQ ID NO: 5 - protein, 7 aa, Artificial
  Belimumab CDRL2
GKNNRPS

SEQ ID NO: 6 - protein, 11 aa, Artificial
  Belimumab CDRL3
SSRDSSGNHWV

SEQ ID NO: 7 - protein, 123 aa, Artificial
  Belimumab VH
QVQLQQSGAEVKKPGSSVRVSCKASGGTFNNNAINWVRQAPGQGLEWMGGIIPMFGTAKYSQNFQGRVAITADESTGTASMELSSLRSEDTAVYYCARSRDLLLFPHHALSPWGRGTMVTVSS

SEQ ID NO: 8 - protein, 109 aa, Artificial
  Belimumab VL
SSELTQDPAVSVALGQTVRVTCQGDSLRSYYASWYQQKPGQAPVLVIYGKNNRPSGIPDRFSGSSSGNTASLTITGAQAEDEADYYCSSRDSSGNHWVFGGGTELTVLG

SEQ ID NO: 9 - protein, 453 aa, Artificial
  Belimumab heavy chain
QVQLQQSGAEVKKPGSSVRVSCKASGGTFNNNAINWVRQAPGQGLEWMGGIIPMFGTAKYSQNFQGRVAITADESTGTASMELSSLRSEDTAVYYCARSRDLLLFPHHALSPWGRGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, Artificial
  Belimumab light chain
SSELTQDPAVSVALGQTVRVTCQGDSLRSYYASWYQQKPGQAPVLVIYGKNNRPSGIPDRFSGSSSGNTASLTITGAQAEDEADYYCSSRDSSGNHWVFGGGTELTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

(19) United States
(12) Patent Application Publication
(10) Pub. No.: US 2023/0220092 A1
(43) Pub. Date: Jul. 13, 2023

(54) Anti-Blys Antibody for Treating Proteinuric Kidney Disease

(71) Applicant: GlaxSmithKline Intellectual Property Management Limited, Brentford, Middlesex (GB)

(72) Inventors: Richard DIMELOW, Stevenage (GB); Herbert STRUEMPER, Triangle Park, NC (US); Debra J. TOMPSO, Stevenage (GB)

(21) Appl. No.: 18/000,893

(22) PCT Filed: Dec. 16, 2020
(86) PCT No.: PCT/EP2020/086589

§ 371 Date: Dec. 6, 2022

Related U.S. Application Data
(60) Provisional application No. 63/038,202, filed on Jun. 12, 2020.

(51) Int. Cl. C07K 16/28; A61P 13/12; A61K 9/00

(52) U.S. Cl. CPC C07K 16/2875; A61P 13/12; A61K 9/0019; A61K 2039/545

(57) ABSTRACT

The present invention relates to an anti-BLyS antibody for use in the treatment of proteinuric kidney disease, for example in the treatment of lupus nephritis. The invention also relates to dosages, duration of treatment and treatment transition for patients moving from intravenous to subcutaneous therapy.

### FIELD OF INVENTION

The present invention relates to a method of treating proteinuric kidney disease in a patient. In particular, a method of treating lupus nephritis using an anti-BLyS antibody. In particular, the invention relates to dosage regimens for administering antibodies that bind to BLyS, such as antagonistic antibodies to BLyS, for use in the treatment of lupus nephritis.

### BACKGROUND TO THE INVENTION

Systemic lupus erythematosus (SLE) is a chronic autoimmune disease characterized by loss of immune tolerance, which can lead to multisystem organ injury. Lupus nephritis (LN) occurs in approximately 40% of patients with SLE and is the most common severe SLE manifestation and a significant cause of morbidity and mortality. To preserve renal function, LN requires fast and effective treatment, yet renal response rates remain unacceptably low, and despite advances in diagnosis and treatment, progression rates at 15 years to end-stage renal disease (ESRD) remain >40% for patients with diffuse proliferative Lupus nephritis. At present, there are no (e.g., US) or limited (e.g., UK, cyclophosphamide/corticosteroid) approved treatment options for LN. Current approaches to the management of LN continue to rely on high dose corticosteroids (HDCS) and broad-spectrum immunosuppressive agents. First line standard therapies include cyclophosphamide (CYC) and HDCS for induction followed by azathioprine (AZA) for maintenance, or mycophenolate mofetil (MMF) and HDCS for induction followed by MMF for maintenance. In addition to the challenges presented by the severity of the disease, cumulative exposure to potent non-selective immunosuppressants and corticosteroid burden are associated with significant side effects, long-term toxicities and organ damage accrual.

A recent meta-analysis of LN outcomes in more than 18,000 LN patients over a 40 year period demonstrated that the rates of progression to ESRD and mortality have not changed appreciably in recent years. This pattern further underlines the limitation of current LN therapies and urgent need for new targeted treatments that can optimize outcomes and improve long-term benefit by reducing kidney inflammation, decreasing LN flares, and delaying progression to ESRD.

Belimumab, a recombinant human IgG1λ monoclonal antibody that inhibits B cell activating factor (BAFF), also known as B lymphocyte stimulator (BLyS), is approved for the treatment of patients ≥5 years of age with active, autoantibody-positive SLE. The efficacy and safety of belimumab in patients with SLE were established in two pivotal phase 3 clinical trials, BLISS-52 and BLISS-76. However, patients with acute, severe LN were excluded from these studies, limiting the investigation of belimumab's effects in active LN.

The registrational belimumab SLE studies excluded subjects with severe active LN because those trials required subjects to be on stable background therapy and had the main outcome measures for overall SLE disease activity. In contrast, patients with active lupus nephritis usually receive more aggressive background therapy and require renal specific assessments in order to be evaluated during a trial. Following the SLE trials and an additional post hoc analysis of patients from them, the BLISS-LN trial was initiated in order to evaluate the efficacy and safety of belimumab plus standard therapy in active LN.

### BRIEF DESCRIPTION OF FIGURES

FIG. 1 shows comparison of Primary Efficacy Renal Response (PERR) by visit (IPD/TF/WD=NR) between the belimumab group and the placebo group.

FIG. 2 shows the time to PERR that is maintained through Week 104 (IPD/TF/WD=NR) for both the belimumab group and the placebo group. The at risk numbers are the number of subjects who have the potential to experience the event at that timepoint. The events are defined as the first event experienced among the following: death, progression to end stage renal disease, doubling of serum creatinine from baseline, renal worsening (reproducible increase in uPCR (to >1 g if the baseline value was <0.2 g, to >2 if the baseline value was between 0.2 g and 1 g, or more than twice the value at baseline if the baseline value was >1 g) or a reproducible decrease in GFR of >20%, accompanied by proteinuria (>1 g), and/or RBC and/or WBC cellular casts), or renal-related treatment failure.

FIG. 3 shows comparison of Complete Renal Response (CRR) by visit (IPD/TF/WD=NR) between the belimumab group and the placebo group.

FIG. 4 shows the time to CRR that is maintained through Week 104 (IPD/TF/WD=NR) for both the belimumab group and the placebo group. The at risk numbers are the number of subjects who have the potential to experience the event at that timepoint. The events are defined as the first event experienced among the following: death, progression to end stage renal disease, doubling of serum creatinine from baseline, renal worsening (reproducible increase in uPCR (to >1 g if the baseline value was <0.2 g, to >2 if the baseline value was between 0.2 g and 1 g, or more than twice the value at baseline if the baseline value was >1 g) or a reproducible decrease in GFR of >20%, accompanied by proteinuria (>1 g), and/or RBC and/or WBC cellular casts), or renal-related treatment failure.

FIG. 5 shows simulations of belimumab serum concentration-time profiles for different baseline proteinuria groups following administration of IV 10 mg/kg q2w to week 4 and q4w thereafter. Solid line black line represents median belimumab concentration; Dark grey shaded area represents interquartile range; Light grey shaded area represents 95% prediction interval.

FIG. 6 shows simulations of belimumab average serum concentrations (C_{avg}) over 4-week time periods following administration of IV 10 mg/kg q2w to week 4 and q4w thereafter for different baseline proteinuria groups. Solid line black line represents median belimumab concentration; Dark grey shaded area represents interquartile range; Light grey shaded area represents 95% prediction interval; Dashed lines represent median and 95% prediction interval of reference <1 g/g uPCR baseline.

FIG. 7 shows simulations of belimumab average serum concentrations (C_{avg}) over 4 week time periods for the >4.5 g/g baseline proteinuria group following administration of IV 10 mg/kg q2w for 4, 8, 12 and 16 weeks followed by q4w dosing thereafter. Solid line black line represents median belimumab concentration; Dark grey shaded area represents interquartile range; Light grey shaded area represents 95% prediction interval; Dashed lines represent median and 95% prediction interval of reference <1g/g baseline.

FIG. 8 shows simulations of belimumab average serum concentrations (C_{avg}) over 4-week time periods following administration of IV 10 mg/kg q2w to week 12 and q4w thereafter for ≥5 g/g uPCR at baseline. Solid line black line represents median belimumab concentration; Dark grey shaded area represents interquartile range; Light grey shaded area represents 95% prediction interval; Dashed lines represent median and 95% prediction interval of reference <1 g/g baseline.

FIG. 9 shows belimumab C_{avg} in PERR non-responders and responders at week 104 stratified by baseline proteinuria. C_{avg} is calculated from weeks 0 to 4 (left panel), weeks 0 to 12 (central panel) and weeks 0 to 24 (right panel). Box plots show the median (solid line), IQR (box), the nearest data point no more than 1.5 times above and below the box (whisker), and outlier data points (points). Subjects who discontinued IP or withdrew from study after the C_{avg} time period were assigned as Week 104 PERR non-responders.

FIG. 10 shows belimumab C_{avg} in CRR non-responders and responders at week 104 stratified by baseline proteinuria. C_{avg} is calculated from weeks 0 to 4 (left panel), weeks 0 to 12 (central panel) and weeks 0 to 24 (right panel). Box plots show the median (solid line), IQR (box), the nearest data point no more than 1.5 times above and below the box (whisker), and outlier data points (points). Subjects who discontinued IP or withdrew from study after the C_{avg} time period were assigned as Week 104 CRR non-responders.

FIG. 11 shows visual predictive check of PERR response stratified by baseline proteinuria (uPCR) above and below 2.5 g/g for the joint dropout/efficacy model.

FIG. 12 shows simulations of belimumab serum concentration-time profiles following administration of subcutaneous (SC) 400 mg q1w to week 4 followed by 200 mg q1w thereafter superimposed on IV 10 mg/kg q2w to week 4 and q4w thereafter (all proteinuria subgroups combined). Solid line represents median belimumab concentration. Shaded area represents 95% prediction interval.

FIG. 13 shows simulations of belimumab serum concentration-time profiles following administration of IV 10 mg/kg q2w for 2 doses followed by SC 200 mg q1w 1 week after the IV dose at week 2 (all proteinuria subgroups combined). Solid black line represents median belimumab concentration; dark grey shaded area represents interquartile range; and light grey represents 95% prediction interval.

FIG. 14 shows simulations of belimumab serum concentration-time profiles following administration of IV 10 mg/kg q2w for 2 doses followed by SC 200 mg q1w 2 weeks after the IV dose at week 2 (all proteinuria subgroups combined). Solid black line represents median belimumab concentration; dark grey shaded area represents interquartile range; and light grey represents 95% prediction interval.

FIG. 15 shows the overall primary efficacy renal response (PERR) and complete renal response (CRR) outcomes at week 104 favoring belimumab over placebo were driven mainly by patients with a proliferative histologic component (Classes III and IV with or without Class V). There was no treatment difference observed for PERR or CRR in patients with pure Class V LN.

FIG. 16 shows the time to a renal-related event or death when stratified by treatment regime and LN class. When stratified by induction regimen used (mycophenolate mofetil [MMF] or cyclophosphamide [CYC]/azathioprine [AZA]) or LN class, the risk of a renal-related event was reduced by belimumab independent of LN class.

FIG. 17 shows the time to renal flares (from Week 24) by treatment regime and LN class, treatment with belimumab reduced the risk of experiencing an LN flare with all standard treatment regimes. The LN flare risk reduction in CYC/AZA-treated patients was greater than in MMF-treated patients.

FIG. 18 shows that when measuring time to a renal-related event or death by baseline proteinuria that belimumab was effective in reducing risk in patients with low and high baseline proteinuria levels.

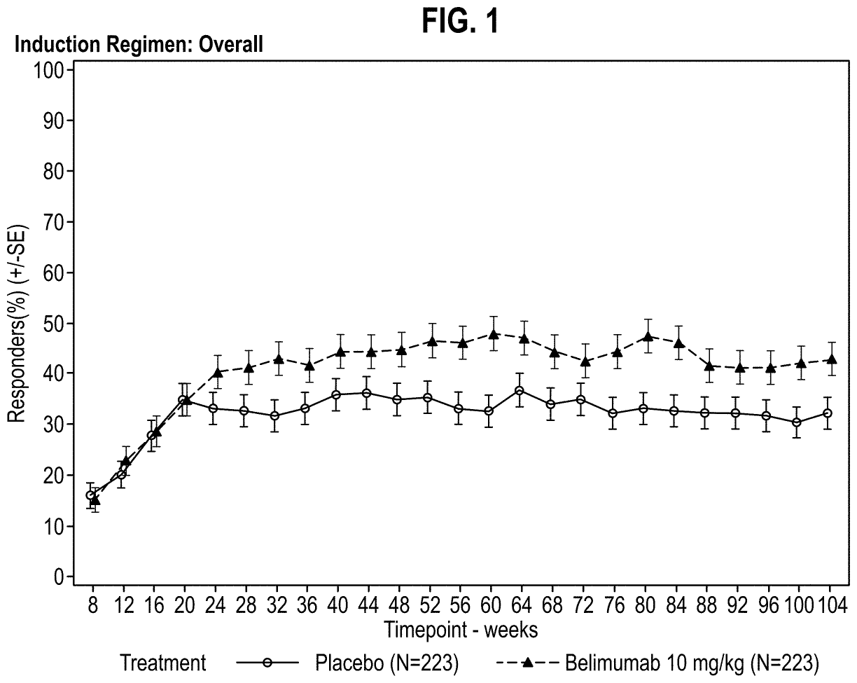

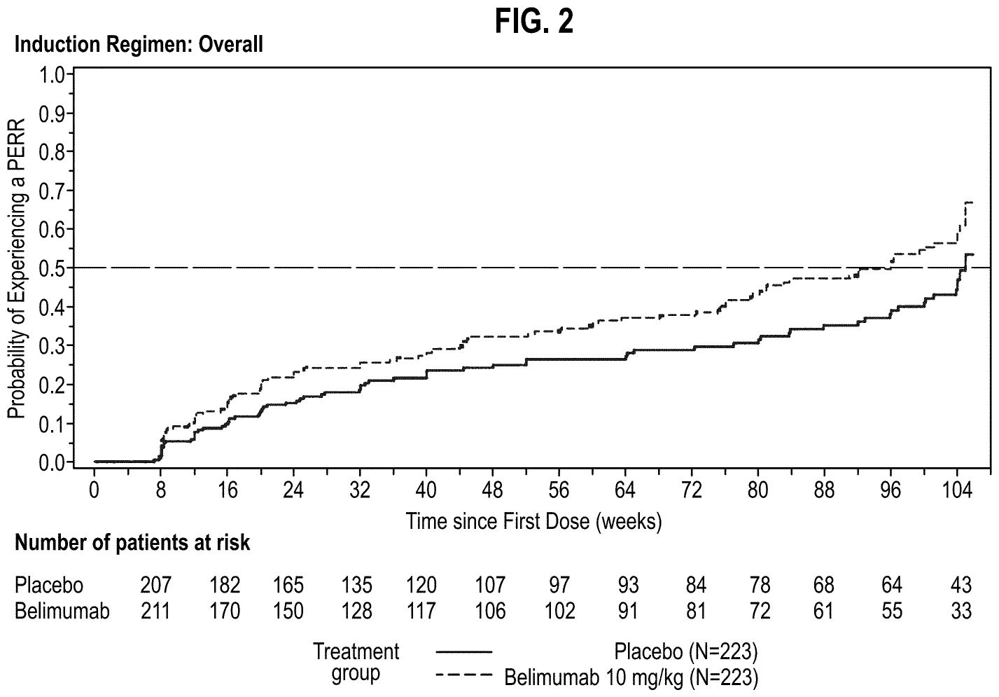

### SUMMARY OF THE INVENTION

In one aspect of the invention as herein described, there is provided an anti-BLyS antibody for use in the treatment of proteinuric kidney disease in a human patient. In one aspect, there is provided an anti-BLyS antibody for use in the treatment of lupus nephritis in a human patient. In one aspect, the use of the anti-BLyS antibody in the treatment is in combination with use of a standard therapy.

In one aspect, there is provided an anti-BlyS antibody for use in preventing or delaying end-stage renal disease in a human patient, for example in a patient with lupus nephritis.

In one aspect, there is provided a method of preventing or treating proteinuric kidney disease in a human patient said method comprising administering an anti-BlyS antibody. In one embodiment, the patient is a lupus nephritis patient.

In one aspect, there is provided a method of preventing or delaying end stage renal disease in a lupus nephritis patient said method comprising administering an anti-BlyS antibody.

In one aspect, there is provided a method of reducing the risk of end-stage renal disease in a patient, said method comprising administering to said patient an effective amount of an anti-BlyS antibody, wherein said patient has at least one characteristic selected from:

(i) proteinuria uPCR<3 g/d,

(ii) eGFR of ≤60 mL/min/1.73 m², and

(iii) biopsy-proven lupus nephritis Class III, IV, and/or with class V.

In one aspect, there is provided a method of reducing the risk of a patient achieving at least a 30% decline in eGFR by administering an anti-BLyS antibody. For example, in one embodiment, there is a decreased risk of a patient having an eGFR more than 30% below the pre-flare level. For example compared to a patient treated with standard therapy alone.

In one aspect, there is an anti-BlyS antibody for use in a method for reducing the rate of decline in kidney function in a patient with lupus nephritis, wherein the patient has a reduction in risk of achieving a 30% decline in eGFR. For example compared to a patient treated with standard therapy alone.

In one aspect, there is provided a method of preserving kidney function in a human, said method comprising administering to said patient an effective amount of an anti-BlyS antibody. For example, there is provided a method of preserving kidney function in a lupus nephritis patient.

In one aspect, there is provided a method of preventing or decreasing renal flares in a patient, said method comprising administering to said patient an effective amount of an anti-BlyS antibody.

In one aspect, there is provided an anti-BLyS antibody for use in the treatment of proliferative lupus nephritis in a human patient. In one embodiment, there is provided an anti-BLyS antibody for use in the treatment of biopsy proven Class III, Class IV, and/or with class V lupus nephritis in a human patient.

In one embodiment, there is provided an anti-BLyS antibody for use in the treatment of membranous lupus nephritis in a human patient.

In one aspect of the invention, there is provided an anti-BLyS antibody for use in the treatment of lupus nephritis wherein the antibody is administered intravenously or subcutaneously to a patient in need thereof.

In one aspect, the anti-BLyS antibody for use in treating lupus nephritis is administered intravenously to a patient in need thereof at a dose of 10 mg/kg every 2 weeks for at least 2 weeks or at least 4 weeks and then every 4 weeks thereafter.

In some patients induction therapy may or may not be required and will be decided by a clinician.

In one aspect, the anti-BLyS antibody for use in treating lupus nephritis is administered subcutaneously to a patient in need thereof at a dose of 200 mg once weekly.

In another aspect, the anti-BLyS antibody for use in treating lupus nephritis is administered subcutaneously to a patient in need thereof at a dose of 400 mg a week for 4 weeks then 200 mg once weekly thereafter.

In another aspect of the invention as herein provided, the anti-BLyS antibody for use in treating lupus nephritis is administered to a human patient who has a proteinuria (uPCR) level of greater than or equal to 4.5 g/g.

In one aspect, the anti-BLyS antibody for use in treating lupus nephritis is administered to a human patient who has a proteinuria (uPCR) level of greater than or equal to 4.5 g/g, wherein the anti-BLyS antibody is administered every 2 weeks for at least the first 12 weeks then every 4 weeks thereafter.

In one aspect, the anti-BLyS antibody for use in treating lupus nephritis is administered to a human patient who has a proteinuria (uPCR) level of greater than or equal to 4.5 g/g and wherein the anti-BLyS antibody is administered at a unit dose of 400 mg a week for at least 12 weeks then 200 mg once weekly thereafter.

In yet another aspect of the invention as herein described, there is provided an anti-BLyS antibody for use in treating lupus nephritis wherein the antibody is administered intravenously at a loading dose of 10 mg/kg every 2 weeks for at least 4 weeks and then subcutaneously at a unit dose of 200 mg per week thereafter.

In yet another aspect, there is provided a method of treating lupus nephritis in a human patient in need thereof comprising administering to said patient a therapeutically effective amount of an anti-BLyS antibody according to the dosage regimens described herein.

In yet another aspect, there is provided a method of reducing proteinuria in a human patient with lupus nephritis, comprising administering to said patient a therapeutically effective amount of the anti-BLyS antibody according to the dosage regimens as described herein.

In yet another aspect, there is provided a method of achieving and maintaining renal response in a human patient with lupus nephritis, comprising administering to said patient a therapeutically effective amount of the anti-BLyS antibody according to the dosage regimens as described herein.

In one aspect, there is a method of treating lupus nephritis in a human patient comprising testing the patient's proteinuria (uPCR) level and if greater than or equal to 4.5 g/g, administering to said patient a therapeutically effective amount of an anti-BLyS antibody intravenously at a dose of 10 mg/kg every 2 weeks for at least the first 12 weeks, followed by every 4 weeks thereafter.

In one aspect, there is a method of treating lupus nephritis in a human patient with a proteinuria (uPCR) level greater than or equal to 4.5 g/g, comprising administering to said patient a therapeutically effective amount of an anti-BLyS antibody intravenously at a dose of 10 mg/kg every 2 weeks for at least the first 12 weeks, followed by every 4 weeks thereafter.

In a further aspect, there is a method of treating lupus nephritis in a human patient comprising testing the patient's proteinuria (uPCR) level and if greater than or equal to 4.5 g/g administering to said patient a therapeutically effective amount of an anti-BLyS antibody subcutaneously at 400 mg a week for at least 12 weeks, followed by 200 mg once weekly thereafter.

In one aspect, there is a method of treating lupus nephritis in a human patient with a proteinuria (uPCR) level greater than or equal to 4.5 g/g, comprising administering to said patient a therapeutically effective amount of an anti-BLyS antibody subcutaneously at 400 mg a week for at least 12 weeks, followed by 200 mg once weekly thereafter.

The invention also provides dosages, duration of treatment and time lapses between administration of the anti-BLyS antibody. Also provided are examples of proteinuric kidney disease to be treated with the invention.

### DETAILED DESCRIPTION OF THE INVENTION

In one aspect of the invention as herein described, there is provided an anti-BLyS antibody, for use in the treatment of proteinuric kidney disease in a human patient. In one embodiment, there is provided an anti-BLyS antibody for use in the treatment of lupus nephritis in a human patient. In one aspect, the use of the anti-BLyS antibody in the treatment is in combination with use of a standard therapy.

In one aspect, there is provided an anti-BlyS antibody for use in preventing or delaying end-stage renal disease in a human patient, for example in a patient with lupus nephritis.

In one aspect, there is provided a method of preventing or treating proteinuric kidney disease in a human patient said method comprising administering an anti-BlyS antibody. In one embodiment, the patient is a lupus nephritis patient.

In one embodiment, there is provided a method of preventing or delaying end-stage renal disease in a lupus nephritis patient said method comprising administering an anti-BlyS antibody.

A method of reducing the risk of end stage renal disease in a patient, said method comprising administering to said patient an effective amount of an anti-BlyS antibody, wherein said patient has at least one characteristic selected from:

(i) proteinuria uPCR<3 g/d,

(ii) eGFR of ≤60 mL/min/1.73 m², and

(iii) biopsy-proven lupus nephritis Class III, IV, and/or with V.

Progressive loss of kidney function in Lupus Nephritis is due to the accumulation of chronic injury which occurs during every renal flare as inflammatory lesions heal and form scars. This chronic injury generally manifests as worsening proteinuria and/or eGFR over time. For example, the time to a decline in eGFR of 30% or 40% are two eGFR milestones considered as predictors of future kidney insufficiency or failure.

In one aspect, there is provided a method of preserving kidney function in a human, said method comprising administering to said patient an effective amount of an anti-BlyS antibody. For example, there is provided a method of preserving kidney function in a lupus nephritis patient.

In one aspect, there is provided a method of preventing or decreasing renal flares in a patient, said method comprising administering to said patient an effective amount of an anti-BlyS antibody.

In one aspect, there is provided a method of reducing the risk of a patient achieving at least a 30% decline in eGFR by administering an anti-BLyS antibody. For example, in one embodiment, there is a decreased risk of a patient having an eGFR more than 30% below the pre-flare level. For example, in one embodiment, there is a decreased risk of a patient having an eGFR more than 40% below the pre-flare level.

In one aspect, there is an anti-BlyS antibody for use in a method for reducing the rate of decline in kidney function in a patient with lupus nephritis, wherein the patient has a reduction in risk of achieving a 30% decline in eGFR.

In one embodiment, the reduction of risk for a patient achieving at least a 30% decline in eGFR is at least 50% or at least 60% or at least 65% or at least 70% compared to the risk for patients treated with placebo. In one embodiment, the reduction of risk for a patient achieving at least a 30% decline in eGFR is at least 50% or at least 60% or at least 65% or at least 70% compared to the risk for patients treated with standard therapy alone.

In one aspect, there is an anti-BlyS antibody for use in a method for reducing the rate of decline in kidney function in a patient with lupus nephritis, wherein the patient has a reduction in risk of achieving a 40% decline in eGFR. In one embodiment, the reduction of risk for a patient achieving at least a 40% decline in eGFR is at least 30% or at least 40% or at least 45% or at least 50% compared to the risk for patients treated with placebo.

In one embodiment, the reduction of risk for a patient achieving at least a 40% decline in eGFR is at least 30% or at least 40% or at least 45% or at least 50% compared to the risk for patients treated with standard therapy alone.

In one aspect, there is provided an anti-BLyS antibody for use in the treatment of proliferative lupus nephritis in a human patient. In one embodiment, there is provided an anti-BLyS antibody for use in the treatment of biopsy proven Class III, Class IV, and/or with Class V lupus nephritis in a human patient. In one embodiment, there is provided an anti-BLyS antibody for use in the treatment of biopsy proven Class III with Class V, Class III with Class IV or Class IV with Class V lupus nephritis in a human patient. In one embodiment, the lupus nephritis patient has biopsy-proven lupus nephritis Class III, IV, and/or with V; and had active renal disease at screening requiring standard therapy.

In one embodiment, there is provided an anti-BLyS antibody for use in the treatment of membranous lupus nephritis in a human patient.

In one aspect, there is provided a method of preserving kidney function in a human, said method comprising administering to said patient an effective amount of an anti-BlyS antibody. For example, there is provided a method of preserving kidney function in a lupus nephritis patient.

The term “antibody” herein is used in the broadest sense and specifically covers monoclonal antibodies, polyclonal antibodies, multispecific antibodies and antibody fragments exhibiting the desired biological activity.

Anti-BlyS binding antibodies of the invention are those which are capable of antagonizing BlyS and may decrease or inhibit BlyS-induced signal transduction. For example, anti-BlyS binding antibodies of the invention may disrupt the interaction between BlyS and its receptor to inhibit or downregulate BlyS-induced signal transduction. In particular, anti-BlyS binding antibodies of the invention which prevent BlyS induced signal transduction by specifically recognizing the unbound BlyS protein, receptor-bound BlyS protein, or both unbound and receptor-bound BlyS protein can be used in accordance with the invention set forth herein. The ability of an anti-BlyS binding antibody of the invention to inhibit or downregulate BlyS induced signal transduction may be determined by techniques known in the art. For example, BlyS-induced receptor activation and the activation of signaling molecules can be determined by detecting the phosphorylation (e.g., tyrosine or serine/threonine) of the receptor or a signaling molecule by immunoprecipitation followed by western blot analysis.

In one embodiment, the anti-BlyS antibody for use according to the invention comprises at least one or more of CDRH1 of SEQ ID NO:1; CDRH2 of SEQ ID NO:2; CDRH3 of SEQ ID NO:3; CDRL1 of SEQ ID NO:4; CDRL2 of SEQ ID NO:5; or CDRL3 of SEQ ID NO:6. In one embodiment, the anti-BlyS antibody for use according to the invention comprises CDRH1 of SEQ ID NO:1; CDRH2 of SEQ ID NO:2; CDRH3 of SEQ ID NO:3; CDRL1 of SEQ ID NO:4; CDRL2 of SEQ ID NO:5; and CDRL3 of SEQ ID NO:6. In a further embodiment, the anti-BlyS antibody for use according to the invention comprises a variable heavy chain sequence of SEQ ID NO: 7 and a light chain variable sequence of SEQ ID NO:8. In a further embodiment, the anti-BlyS antibody for use according to the invention comprises a heavy chain sequence of SEQ ID NO:9 and a light chain sequence of SEQ ID NO:10. In a further embodiment the anti-BlyS antibody for use according to the invention is belimumab.

In one aspect of the invention, the anti-BlyS antibody for use in the treatment of a proteinuric kidney disease such as lupus nephritis is administered intravenously. In one embodiment, the anti-BlyS antibody is administered at a dose of 10 mg/kg. In a further embodiment, the anti-BlyS antibody is administered every 2 weeks, which means that the anti-BlyS antibody is administered at 2 week intervals, for example 3 doses in 4 weeks at day 0, day 14 and day 28. In a further embodiment, the anti-BlyS antibody is administered every 2 weeks (i.e., following administration at day 0) for at least 4 weeks, or for at least 6 weeks or for at least 8 weeks and then every 4 weeks thereafter.

In one aspect of the invention, the anti-BlyS antibody for use in the treatment of a proteinuric kidney disease such as lupus nephritis is administered subcutaneously. Subcutaneous injections of the present invention may be administered as single injections wherein the entire dose is administered as a single shot, wherein the entire volume of the dose is administered all at once. A single shot injection may be administered multiple times. A single shot differs from a continuous or titrated administration, e.g. an infusion, wherein the administration may be administered over several minutes, hours or days until a full dose is achieved.

In one embodiment, the anti-BlyS antibody is administered at a unit dose of 200 mg. In a further embodiment, the anti-BlyS antibody is administered once every week. In a further embodiment, the anti-BlyS antibody is administered twice a week at predominantly the same time point, for example within the same hour or for example the same day. In one embodiment, the anti-BlyS antibody is administered at a total dose of 400 mg. In a further embodiment the 400 mg dose may be provided by more than one injection, for example a 200 mg unit dose is administered twice. The anti-BlyS antibody may be administered at the same or different injection sites but is preferably administered at different injection sites. In a further embodiment, the anti-BlyS antibody is administered twice weekly, sequentially or concomitantly, at different reaction sites. In yet a further embodiment, the anti-BlyS antibody is administered at a dose of 400 mg a week for 4 weeks, for example at day 0, day 7, day 14, day 21, and day 28, and then at a dose of 200 mg once weekly thereafter. In another embodiment the anti-BlyS antibody is administered at a dose of 400 mg a week (i.e. following administration at day 0) for at least 4 weeks, or for at least 8 weeks or for at least 12 weeks, and then at a dose of 200 mg once weekly thereafter.

In one aspect, there is provided an anti-BlyS antibody for use in a patient with biopsy confirmed active lupus nephritis that is autoantibody positive.

In another aspect of the invention, the human patient has a proteinuria level of at least 1 g/g (uPCR≥1 g/g) prior to treatment. Proteinuria is the leakage of protein from the blood into the urine. In a healthy kidney, when the body eliminates waste, protein is kept in the blood stream because the protein in the blood is too large to pass through the tiny holes in the kidney filters. However, as lupus nephritis causes damage to the kidneys more protein is able to pass through into the urine giving an indication of renal disease. Proteinuria is defined as a ratio of protein/creatinine (uPCR value). In a further embodiment, the human patient has a proteinuria level of at least 2 g/g (uPCR≥2 g/g) prior to treatment, or at least 3 g/g (uPCR≥3 g/g) prior to treatment, or at least 3.5 g/g (uPCR≥3.5 g/g) prior to treatment, or at least 4 g/g (uPCR≥4.5 g/g) prior to treatment, or at least 5 g/g (uPCR≥5 g/g) prior to treatment.

As the renal impact of lupus nephritis leads to increased proteinuria the exposure benefit from the anti-BlyS antibody may be lost and as such appropriate dose adjustment may be required to maintain an average concentration of antibody (C_{avg}) at steady state.

In one embodiment of the invention, there is provided an anti-BlyS antibody for use in the treatment of lupus nephritis in a human patient wherein the human patient has a proteinuria level of at least 3 g/g (uPCR≥3 g/g), or at least at least 4 g/g (uPCR≥4 g/g), or at least 4.5 g/g (uPCR≥4.5 g/g), or at least 5 g/g (uPCR≥5 g/g) prior to treatment and wherein the anti-BlyS antibody is administered intravenously at a dose of 10 mg/kg every 2 weeks for at least the first 8 week or at least the first 10 week or at least the first 12 weeks or at least the first 16 weeks or at least the first 20 weeks and then every 4 weeks thereafter.

In one embodiment, the anti-BlyS antibody is administered at a dose of 10 mg/kg every 2 weeks for at least the first 12 weeks and then every 4 weeks thereafter.

In one embodiment of the invention, there is provided an anti-BlyS antibody for use in the treatment of lupus nephritis in a human patient wherein the human patient has a proteinuria level of at least 3 g/g (uPCR≥3 g/g), or at least at least 4 g/g (uPCR≥4 g/g), or at least 4.5 g/g (uPCR≥4.5 g/g), or at least 5 g/g (uPCR≥5 g/g) prior to treatment and wherein the anti-BlyS antibody is administered subcutaneously at a dose of 400 mg a week or a unit dose of 200 mg twice weekly for a period of at least 6 weeks, or for at least 8 weeks, or for at least 12 weeks or for at least 16 weeks and then 200 mg once weekly thereafter. In one embodiment, the anti-BlyS antibody is administered subcutaneously at a dose of 400 mg a week for a period of at least 12 weeks and then 200 mg once weekly thereafter.

It may be necessary or beneficial over time to move patients from intravenous administration to subcutaneous administration. Therefore, in one aspect of the invention, there is provided an anti-BlyS antibody for use according to the present invention wherein the anti-BlyS antibody is administered intravenously prior to subcutaneous administration. In one embodiment, the anti-BlyS antibody is administered intravenously at a loading dose of 10 mg/kg for at least 1 week prior to subcutaneous administration. In a further embodiment the anti-BlyS antibody is administered intravenously at a loading dose of 10 mg/kg every 2 weeks for at least 2 weeks, at least 4 weeks, or at least 6 weeks and then subcutaneously at a unit dose of 200 mg per week thereafter. In a further embodiment, the anti-BlyS antibody is administered intravenously at a loading dose of 10 mg/kg every 2 weeks for at least 2 weeks and then subcutaneously at a unit dose of 200 mg per week thereafter.

In one aspect, the anti-BlyS antibody for use according to the invention is administered to a human patient in need thereof to maintain a systemic exposure steady-state average concentration (C_{avg}) of 90-120 μg/mL. In one embodiment, the C_{avg} is maintained at 100-110 μg/mL at steady state. In a further embodiment, the C_{avg} is maintained at 100-105 μg/mL at steady state.

In one aspect, the anti-BlyS antibody for use according to the invention is administered as a monotherapy or in combination with other therapies. In one embodiment, the anti-BlyS antibody is co-administered with standard of care medicaments such as, for example, High Dose Corticosteroids (HDCS), Cyclophosphamide (CYC), Azathioprine (AZA) and/or Mycophenolate Mofetil (MMF).

In one embodiment, the anti-BlyS antibody is co-administered with High Dose Corticosteroids (HDCS) and Cyclophosphamide (CYC) for induction therapy followed by Azathioprine (AZA) for maintenance therapy; or HDCS and Mycophenolate Mofetil (MMF) for induction therapy followed by MMF for maintenance therapy.

In a further embodiment, the induction therapy is started within 60 days of the first dose of the anti-BlyS antibody.

In another embodiment, the anti-BlyS antibody may be combined with other biologics or therapeutics such as other antibodies or therapies, such as voclosporin. In one embodiment, the anti-BlyS antibody is used in combination with voclosporin. The voclosporin used is preferably a mixture of greater than about 80% E isomer and less than about 20% Z isomer, and more preferably greater than about 90% E isomer and less than about 10% Z isomer. In one embodiment, daily dosages of voclosporin over a projected period of 24, 48, 52 weeks or longer are employed, wherein the voclosporin is administered twice daily (BID). Suitable dosages are in increments of 7.9 mg, including 39.5 mg, 31.6 mg, 23.7 mg, 15.8 mg or 7.9 mg. Low dosages show superior results compared to a higher dose of 39.5 mg and each of such administrations is carried out twice daily. Doses as low as 15.8 mg or 7.9 mg twice daily are effective. Varying dosages for use in combination are also considered.

It is also advantageous and part of the invention to evaluate a subject who has been treated with the dosage regimens of the present invention at the end of the treatment period (e.g. 104 weeks) to determine whether a complete or partial remission has occurred. Further evaluations are included at a time subsequent to termination of the treatment to assess whether the remission achieved according to the measurement at the end of the treatment is being maintained. Such evaluation may also be done at intermediate times during treatment to determine whether dosage can be reduced or increased either in concentration or frequency.

In one aspect, the anti-BlyS antibody for use according to the invention is administered to a human patient in need thereof for a period of at least 52 weeks or for at least 104 weeks and the patient has a higher chance to achieve the following results compared to a patient receiving placebo:
i) a Glomerular Filtration Rate (eGFR)≥60 mL/min/1.73 m² or has an eGFR no more than 20% below the pre-flare value; and/or
ii) a urinary protein:creatinine ratio (uPCR)≤0.7.

In one aspect, the anti-BlyS antibody for use according to the invention is administered to a human patient in need thereof for a period of at least 52 weeks or for at least 104 weeks and the patient has a higher chance to achieve the following results compared to a patient receiving placebo:
i) a Glomerular Filtration Rate (eGFR)≥90 mL/min/1.73 m² or has an eGFR no more than 10% below the pre-flare value; and/or
ii) a urinary protein:creatinine ratio (uPCR)≤0.5.

In one embodiment, the human patient has a 5%, 6%, 7%, 8%, 9%, 10%, 11% or 12% higher chance of achieving the results described herein. In yet a further embodiment the human patient has a 5-10% higher chance for achieving the results described herein. In one embodiment, the human patient has at least a 10% higher chance of achieving the results described herein compared to a patient on placebo or on standard therapy alone. In yet a further embodiment the human patient achieves the results described herein after 52 weeks and/or after 104 weeks of treatment. In one embodiment, the results are defined as:
i) a Glomerular Filtration Rate (eGFR)≥60 mL/min/1.73 m² or has an eGFR no more than 20% below the pre-flare value; and
ii) a urinary protein:creatinine ratio (uPCR)≤0.7.

In a further embodiment, the results are defined as:
i) a Glomerular Filtration Rate (eGFR)≥90 mL/min/1.73 m² or has an eGFR no more than 10% below the pre-flare value; and
ii) a urinary protein:creatinine ratio (uPCR)≤0.5.

In one aspect, there is therefore provided a method of treating a proteinuric kidney disease, such as lupus nephritis, which method comprises administering to a subject diagnosed with said disease a predetermined dosage of effective amounts of anti-BlyS antibody over a projected treatment period of at least 52 weeks or at least 104 weeks, said method further comprising.
(a) assessing the estimated Glomerular Filtration Rate (eGFR) of said subject at at least a first time point and a second time point on different days of said treatment period, and
(b)
(i) if the eGFR of said subject decreases by more than a target % to below a predetermined value between said first and second time points, increasing the dosage to said subject;
(ii) if the eGFR of said subject decreases by less than said target % between said first and second time points, continuing administering the same predetermined dosage of anti-BlyS antibody to said subject.

Critical parameters used to assess the desirability of dosage adjustment is the eGFR rate and uPCR level. Chronic kidney disease is sometimes defined as eGFR as ≤60 mL/min/1.73 m² for more than three months without kidney damage or above this level with kidney damage.

As noted above, a further decrease in eGFR is a negative side effect that may occur during treatment.

Typically, a baseline value of the eGFR is established either at the beginning of the protocol or at some “first time point” during the protocol. If the decrease is greater than a target percentage, which is typically between 20%-45% (e.g., 20%, 25%, 30%, 35%, 40%, or 45%) as compared to the first time point, a dose adjustment may be necessary. If the decrease is less than that target percentage, maintenance of treatment at the same level as indicated.

In one embodiment, the method further comprises evaluating said subject for renal function at a time point after the end of said treatment period by assessing eGFR and/or protein/creatinine ratio (uPCR).

In one aspect, there is provided a method of treating proteinuric kidney disease according to the invention as described herein. Proteinuric Kidney disease may include lupus nephritis, diabetic nephropathy, nephrotic syndromes (i.e. intrinsic renal failure), nephritic syndromes, toxic lesions of kidneys, glomerular diseases, such as membranous glomerulonephritis, focal segmental glomerulosclerosis (FSGS), IgA nephropathy (i.e., Berge's disease), IgM nephropathy, membranoproliferative glomerulonephritis, membranous nephropathy, minimal change disease, hypertensive nephrosclerosis and interstitial nephritis. In one embodiment, the proteinuric kidney disease is lupus nephritis.

In yet another aspect, there is provided a method of treating lupus nephritis in a human patient comprising the steps of:
i) [optionally] obtaining a sample from said human patient;
ii) testing of the sample for proteinuria (uPCR) level;
iii) if proteinuria (uPCR) level is greater than or equal to 3.0 or 4.0 or 4.5 or 5.0 g/g, then administering an anti-BLyS antibody either:

a) intravenously at a dose of 10 mg/kg every 2 weeks for at least the first 12 weeks then every 4 weeks thereafter; or

b) subcutaneously at a dose of 400 mg a week for a period of at least 12 weeks then 200 mg once weekly thereafter.

In yet another aspect, there is provided a method of treating lupus nephritis in a human patient with a proteinuria (uPCR) level greater than or equal to 3.0 or 4.0 or 4.5 or 5.0 g/g, comprising administering an anti-BLyS antibody either:

a) intravenously at a dose of 10 mg/kg every 2 weeks for at least the first 12 weeks then every 4 weeks thereafter; or

b) subcutaneously at a dose of 400 mg a week for a period of at least 12 weeks then 200 mg once weekly thereafter.

In yet another aspect, there is provided a method of treating a proteinuric kidney disease which method comprises administering to a subject diagnosed with said disease a predetermined dosage of an effective amount of an anti-BLyS antibody over a projected treatment period of at least 12 weeks, said method further comprising:

(a) assessing the proteinuria (uPCR) level of said subject before the treatment period, and

(b) if the uPCR of said subject is higher than or equal to 3.0 or 4.0 or 4.5 or 5.0 g/g, treating the subject by administering an anti-BLyS antibody either:

i) intravenously at a dose of 10 mg/kg every 2 weeks for at least the first 12 weeks then every 4 weeks thereafter; or

ii) subcutaneously at a unit dose of 200 mg twice weekly for a period of at least 12 weeks then 200 mg once weekly thereafter.

A primary end point used in clinical analysis is primary efficacy renal response (PERR), defined as urinary protein creatinine ratio≤0.7, estimated glomerular filtration rate (eGFR) not more than 20 percent (%) below the pre-flare value or ≥60 millilitres per minute per 1.73 square meter (mL/min/1.73 m²) and where not a treatment failure. In one embodiment of the invention, there is provided at least a 40% or at least a 45% or at least 50% higher chance of achieving a PERR when administered belimumab than placebo or when treated with standard therapy alone. In one embodiment, there is provided at least a 46% higher chance of achieving a PERR when administering belimumab than placebo or when treated with standard therapy alone.

Secondary endpoints used in clinical analysis include reaching PERR at a shorter time frame, such as after 52 weeks of treatment and may be maintained to at least 104 weeks of treatment;

Complete renal response (CRR), defined as urinary protein creatinine ratio<0.5, eGFR not more than 10% below the pre-flare value or ≥90 mL/min/1.73 m² and where not a treatment failure;

Time to a renal-related event. A renal-related event is defined as any of the following: i) end-stage renal disease, ii) doubling of serum creatinine, iii) renal worsening from baseline or iv) renal disease-related treatment failure; or

Ordinal response rate (ORR) is defined with respect to reproducible responses that included complete response rate (CRR), partial response rate (PRR) and non-responder. In one embodiment of the invention, there is provided at least a 50% or at least a 55% or at least 60% higher chance of achieving a CRR when administered belimumab than placebo or when administered standard therapy alone. In one embodiment, there is provided at least a 58% higher chance of achieving a CRR when administering belimumab than placebo or when administered stabdarda therapy alone.

The invention is now exemplified with reference to the following non-limiting examples.

### EXAMPLES

### Example 1

### Clinical Study Population

Study BEL114054 was designed to address the high unmet need in LN and to support an indication for belimumab in LN. The study evaluated the efficacy and safety of belimumab added to a range of standard therapies for induction and maintenance of renal response and prevention of renal worsening in adult subjects with active LN. The study enrolled a wide range of LN subjects across different regions, races, LN classes, and standard of care therapies. As a result, the study subjects represented the real-world patients with active LN.

A total of 448 subjects were randomized in the study, 223 in each group were included in the modified intent to treat population. Treatment was stratified by induction regimen and race. Treatment was completed as scheduled to the Week 100 visit by 62.3% subjects (59.2% placebo, 65.5% belimumab) and the study was completed through Week 104 by 79.6% subjects (75.8% placebo, 83.4% belimumab).

Demographic and baseline characteristics were generally balanced between the treatment groups, and comparable to the LN populations reported in the literature. When compared to the pooled belimumab SLE population, the subjects enrolled in the BEL114054 study were younger, with shorter SLE duration and higher clinical and serological SLE activity. Just above 40% of the study population had nephrotic range proteinuria at baseline (uPCR≥3 g/g), while in the pivotal Phase 3 SLE studies, 6% of subjects had uPCR≥2 g/g. In contrast to the SLE population in the belimumab studies, all subjects in BEL114054 were receiving potent immunosuppressants and high doses of corticosteroids for induction of LN remission at baseline.

### Example 2

### Clinical End Points

The PERR represents a clinically relevant composite endpoint including 3 key components: proteinuria improvement to a level predictive of positive long-term renal outcome, no clinically significant decline in GFR, and no need for prohibited medications that are taken for renal worsening or can affect key renal outcomes. The major secondary endpoints included renal response measures that represent greater magnitude of renal improvement based on a more stringent response criteria (CRR at Week 104), earlier renal response (PERR at Week 52) and effect on preventing renal events associated with poor long-term prognosis (time to renal-related event or death endpoint). For both PERR and CRR the response over time and time to response maintained through Week 104 were evaluated for onset and durability of renal response.

In addition to evaluation of renal efficacy, measures of the overall SLE activity [assessed by SLEDAI-S2K and SLE Flare Index; SFI] were included to evaluate effects of belimumab in the context of a systemic disease treatment.

To ensure clinically relevant assessment of belimumab efficacy in a population with high corticosteroid burden at baseline, mandatory corticosteroid taper to mg/day of prednisone equivalent was required to be completed by Week 24. Subjects who exceeded corticosteroid dose of 10 mg/day from 24 weeks apart from the protocol allowable treatment were considered treatment failure for evaluation of the key efficacy endpoints. This design feature helped to address two important points: masking effects of corticosteroids on potential benefits of belimumab treatment; and provision of evidence supporting steroid burden reduction in the context of improved renal outcomes.

Subjects who discontinued investigational agent treatment for any reason were encouraged to remain in the study until the end of the double-blind treatment period at Week 104. This approach allowed for the efficacy and safety data collection regardless of the investigational agent continuation status to support treatment policy analyses.

### Example 3

### Clinical Relevance of Key Renal Efficacy Outcome

Primary Efficacy Renal Response

The goal of LN management is to improve renal survival, therefore the definite endpoint to test novel treatments' effects would be development of End stage renal disease (ESRD) or the need for renal replacement therapy. However, such events occur infrequently and take a long time to develop, hence, making placebo-controlled clinical studies designed around these endpoints not feasible. To overcome this challenge, many clinical trials use surrogate endpoints of renal response, but because there is no consensus on the definition of the response, the endpoints used in previous LN clinical trials lacked uniformity and whether they reflected long-term kidney survival is questionable.

Lack of uniform guidelines on short-term prognostic factors predictive of long-term outcomes in LN makes it challenging to adjust the intensity and duration of LN treatment in clinical practice, and to provide consistent and accurate evaluation of novel treatments in clinical trials.

The primary endpoint (PERR) in BEL114054 represents a binary, easily interpretable composite outcome that includes assessment of proteinuria and GFR (Glomerulat filtration rate). The PERR components and their definitions of response are based on the latest scientific knowledge in LN.

Both proteinuria and GFR are measured in clinical practice and serve as key indicators of development and progression of kidney disease. Therefore, both parameters should be used in the composite endpoint of renal response. Urinary sediment was not included as a component, because of the evidence suggesting that it does not provide additional value and, in fact, may confound the results. Proteinuria is a marker of renal damage and plays an important role in pathogenesis of kidney disease. Proteinuria reduction to <0.7-0.8 g/day at 12 months as a result of LN treatment had been initially reported as the best single predictor of good long-term renal prognosis in 2 previous studies evaluating LN progression and outcomes. The results from these studies of predominantly Caucasian patients with newly diagnosed LN were later replicated in 2 independent ethnically/racially diverse LN cohorts, including the largest cohort to date, comprising 484 LN patients. Proteinuria reduction to <0.5 g/g may not be always achievable in patients with advanced LN due to irreversible renal damage, although it does not mean lack of response to treatment and poor prognosis. The proteinuria cut off <0.5 g/g may have been too restrictive to predict many good renal outcomes, as reported in the literature.

Changes in proteinuria alone can be a reliable marker of long-term prognosis in newly diagnosed LN with relatively intact renal function, although in a more advanced disease, substantial decrease in GFR serves as an independent marker of progressive nephron loss and unfavourable future outcomes. GFR<60 mL/min/1.73 m² is established as one of the chronic kidney disease (CKD) definition criteria. Such GFR decrease even in the absence of proteinuria is a risk factor of poorer long-term prognosis due to increased CKD complications, more rapid progression to ESRD and mortality.

End Stage Renal Disease (ESRD) is the final, permanent stage of chronic kidney disease, where kidney function has declined to the point that the kidneys can no longer function on their own. ESRD is often defined as an eGFR of <30 mL/min/1.73 m² or <15 mL/min/1.73 m² and/or requiring dialysis.

To explore if meeting the uPCR and eGFR criteria of PERR at 24 months is associated with better long-term outcomes, a retrospective analysis of the 173 LN subjects from the Hopkins Lupus cohort was performed. The results demonstrated that achieving PERR at 24 months post biopsy was significantly associated with lower likelihood of CKD development (HR: 0.26, 95% CI: 0.14, 0.47; p<0.0001) and progression to ESRD/mortality (HR: 0.33, 95% CI: 0.13, 0.87; p=0.0255) in subjects with LN, therefore supporting the predictive value of PERR.

In addition to the uPCR and GFR components of renal response, intake of prohibited medications resulting in treatment failure is an important component of efficacy evaluation in the clinical trial setting. Firstly, absence of restrictions on concomitant medications use can significantly confound the results and mask true effects of an investigational treatment compared to placebo. Secondly and more importantly, in most cases concomitant medications that affect uPCR and eGFR evaluation (e.g. immunosuppressants and corticosteroids) are taken because of insufficient LN control, therefore representing failure to respond to the assigned LN treatment. For these reasons, PERR and other key efficacy endpoints include need for prohibited medications resulting in treatment failure as a reason for non-response.

Time to Renal-Related Event or Death

Time to renal-related event or death is a composite endpoint that evaluates effects of treatment on the long-term renal outcomes and their predictors. The renal-related event includes ESRD, doubling of serum creatinine from baseline, renal worsening (defined by increased proteinuria and/or impaired renal function), or renal-related treatment failure. This is a highly clinically relevant outcome that in contrast to renal response, is less confounded by proteinuria and focuses on what is most important to the patients and health care providers in the context of living with and managing a life-long serious condition.

Death and ESRD are ultimate clinical outcomes that define the LN prognosis. These are expected to occur at a low rate during the treatment period.

Additional components of the endpoint include the events associated with increased risk of progression to ESRD and overall mortality.

Sustained doubling of serum creatinine from baseline—an indicator of accumulation of renal damage resulting in progressive nephron loss;

Renal worsening/flare (defined by worsening in proteinuria and/or eGFR)—associated with significant renal damage accrual per each episode and cumulative drug toxicities related to aggressive treatment.

Intake of protocol prohibited treatments for inadequate LN control or renal flare management—indicates failure to respond to treatment, therefore more refractory disease with faster renal damage accrual and accumulation of treatment-related toxicities.

### Example 4

### Efficacy Results for Lupus Nephritis Study BEL114054

BEL114054 demonstrated statistical significance in favour of the belimumab group for each of the efficacy endpoints in the pre-specified testing sequence (Table 1).

Importantly, all subjects were receiving high doses of corticosteroids at the time of randomization and improvements in efficacy outcomes with belimumab treatment were demonstrated on a background of mandatory corticosteroid dose reduction to 10 mg/day or less that applied from Week 24 onwards.

TABLE 1
Primary and Major Secondary Efficacy Summary Table (Double-Blind, mITT)

| | | | Observed | Odds/Hazard | |
|---|---|---|---|---|---|
| | | Belimumab | difference | ratio (95% | |
| | Placebo | 10 mg/kg | (%) vs. | CI)^{a} vs. | p- |
| Efficacy Endpoint | N = 223 | N = 223 | placebo | placebo | value^{a} |
| PERR at Week 104^{a} | | | | | |
| Responders, n (%) | 72 (32.3) | 96 (43.0) | 10.76 | OR = 1.55 | 0.0311 |
| | | | | (1.04, 2.32) | |
| Components of PERR (%) | | | | | |
| Urine protein:creatinine ratio | 33.6 | 44.4 | 10.76 | OR 1.54 | 0.0320 |
| ≤0.7 | | | | (1.04, 2.29) | |
| eGFR ≥60 mL/min/1.73 m² or | 50.2 | 57.4 | 7.17 | OR 1.32 | 0.1599 |
| no more than 20% below | | | | (0.9, 1.94) | |
| pre-flare value | | | | | |
| Not treatment failure^{b} | 74.4 | 83.0 | 8.52 | OR 1.65 | 0.0364 |
| | | | | (1.03, 2.63) | |
| CRR at Week 104^{a} | | | | | |
| Responders, n (%) | 44 (19.7) | 67 (30.0) | 10.31 | OR = 1.74 | 0.0167 |
| | | | | (1.11, 2.74) | |
| Components of CRR (%) | | | | | |
| Urine protein:creatinine ratio | 28.7 | 39.5 | 10.76 | OR 1.58 | 0.0268 |
| <0.5 | | | | (1.05, 2.38) | |
| No decrease in eGFR from | 39.9 | 46.6 | 6.73 | OR 1.33 | 0.1539 |
| pre-flare of >10% (or within | | | | (0.90, 1.96) | |
| normal range: ≥90 | | | | | |
| mL/min/1.73 m²) | | | | | |
| Not treatment failure^{b} | 74.4 | 83.0 | 8.52 | OR 1.65 | 0.0364 |
| | | | | (1.03, 2.63) | |
| PERR at Week 52^{a} | | | | | |
| Responders, n (%) | 79 (35.4) | 104 (46.6) | 11.21 | OR = 1.59 | 0.0245 |
| | | | | (1.06, 2.38) | |
| Time to Renal-Related | | | | | |
| Event or Death^{c} | | | | | |
| Percentage of subjects with | 63 (28.3) | 35 (15.7) | — | HR = 0.51 | 0.0014 |
| event | | | | (0.34, 0.77) | |
| Ordinal Renal Response | | | | | |
| at Week 104^{d}, n (%) | | | | | |
| Complete Renal Responders | 44 (19.7) | 67 (30.0) | 10.31 | — | 0.0096 |
| Partial Renal Responders | 38 (17.0) | 39 (17.5) | 0.45 | — | |
| Non-Responders | 141 (63.2) | 117 (52.5) | −10.76 | — | |

OR = Odds Ratio, HR = Hazard Ratio
^{a}Odds Ratio (95% confidence interval) and p-value are from a logistic regression model for the comparison between belimumab and placebo with covariates treatment group, induction regimen (CYC vs. MMF), race (black vs. non-black), baseline uPCR, and baseline eGFR. Study Withdrawal (WD), Treatment Failures (TF) and Investigational Product Discontinuation (IPD) are imputed as non-responders.
^{b}Treatment failure: Subject who took a protocol-prohibited or restricted medication or dose.
^{c}Events are defined as the first event experienced among the following: death, progression to end stage renal disease, doubling of serum creatinine from baseline, renal worsening (reproducible increase in uPCR (to >1 g if the baseline value was <0.2 g, to >2 if the baseline value was between 0.2 g and 1 g, or more than twice the value at baseline if the baseline value was >1 g) or a reproducible decrease in GFR of >20%, accompanied by proteinuria (>1 g), and/or RBC and/or WBC cellular casts), or renal-related treatment failure. Subjects who discontinue randomized treatment, withdraw from the study, or are lost to follow-up are censored on the date of the event. Subjects who complete the 104-week treatment period are censored at the Week 104 visit. Time to event is defined as (event date-treatment start date + 1). From Cox proportional hazards model for the comparison between Belimumab and Placebo adjusting for induction regimen, race, baseline uPCR, and baseline eGFR.
^{d}P-value is from a rank analysis of covariance (rank ANCOVA) model comparing belimumab and placebo with covariates for treatment group, induction regimen (CYC vs MMF), race (black vs non-black), baseline uPCR, and baseline eGFR. Study Withdrawal (WD), Treatment Failures (TF) and Investigational Product Discontinuation (IPD) are imputed as non responders.

### Example 5

### PERR and Supportive Analyses

The PERR response rate at Week 104 was significantly higher in the belimumab group compared with the placebo group. As seen in Table 1, for the response for individual components of PERR, the largest treatment effect in favour of belimumab was observed in the uPCR component. Fewer subjects in the belimumab group met treatment failure definition as a result of use of prohibited therapies, such as increase in corticosteroid dose and/or initiation of a new immunosuppressant due to renal disease worsening.

When PERR was analysed over time, numerically higher response rates in the belimumab group compared with the placebo group were demonstrated across all timepoints from Week 24 onwards (Error! Reference source not found.).

The durability of PERR response was evaluated by analysing time to PERR maintained through Week 104. There was a 46% increased chance of achieving a PERR that would be maintained to Week 104 at any timepoint in the belimumab group compared with the placebo group (HR 1.46; 95% CI: 1.07, 1.98; p=0.0157) (Error! Reference source not found.).

The probability of having started a response that lasted through Week 104 was higher in the belimumab group from the first timepoint onwards. Earlier separation in achieving durable PERR suggests that early responses in the placebo group were less likely to be sustained than in the belimumab group.

To evaluate earlier renal response in the study, PERR was analysed after 52 weeks of treatment (second major secondary endpoint). The results of this analysis were consistent with the Week 104 results demonstrating statistically significant superiority of belimumab (Table 1).

### Example 6

### CRR and Supportive Analyses

The number of CRR responders at Week 104 was significantly higher in the belimumab group compared with the placebo group and all 3 components consistently favoured belimumab. Belimumab superiority demonstrated for this more stringent endpoint reinforces the robustness of the efficacy evidence. Similar to the PERR components results, the largest treatment effect in favour of belimumab was observed in the uPCR CRR component (Table 1). The same treatment failure rules applied for the PERR and CRR endpoints, therefore, the treatment failure rates were lower on belimumab treatment as compared with placebo.

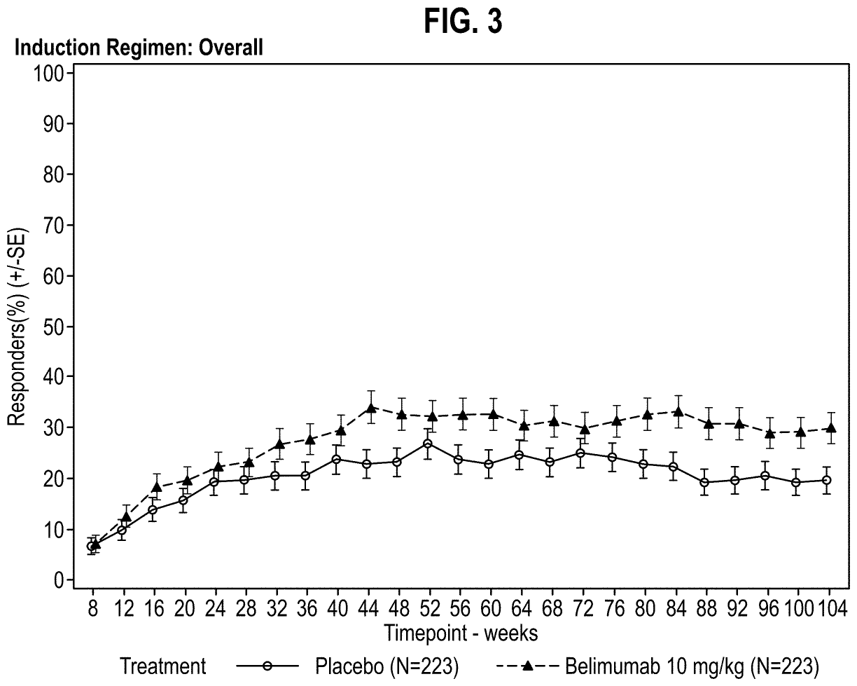

In support of the CRR at Week 104 results, the response rates over time showed that beginning at Week 12, a numerically greater percentage of subjects receiving belimumab achieved CRR compared with placebo and this difference was maintained through to Week 104 (FIG. 3).

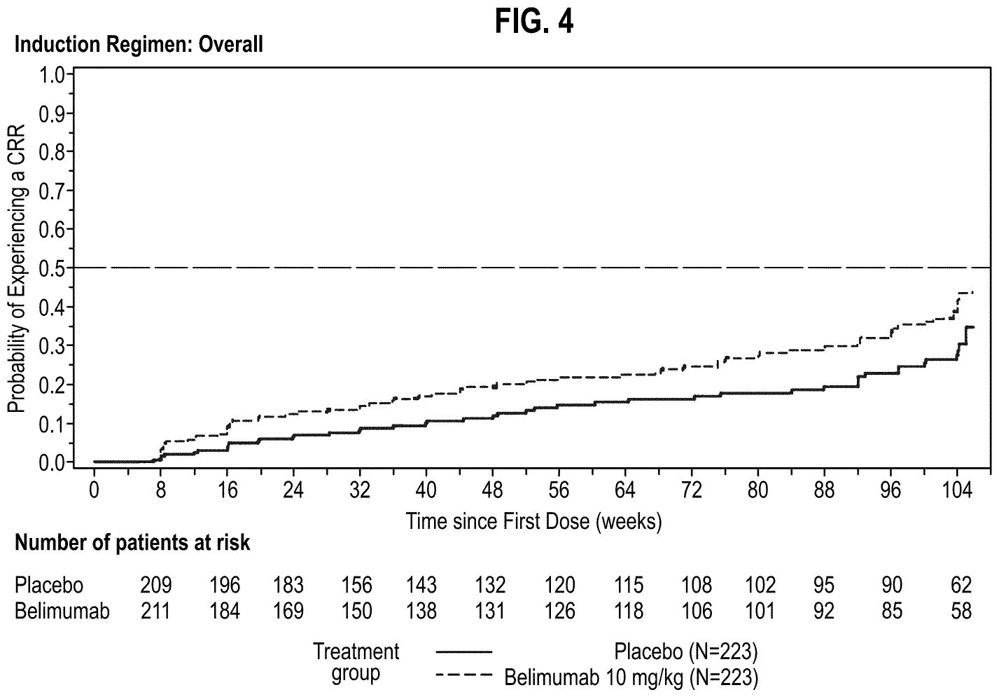

There was a 58% increased chance of achieving a CRR that would be maintained to Week 104 in the belimumab group compared with the placebo group (HR 1.58; 95% CI: 1.08, 2.31; p=0.0189) (FIG. 4). Consistent with the PERR results, the probability to achieve durable CRR early was higher for belimumab-treated subjects as compared to placebo.

### Example 7

### Multivariable Analyses

Multivariable Analysis of PERR at Week 104

A multivariable logistic regression analysis was performed using the data from BEL114054 to identify baseline factors that are predictive of PERR at Week 104 irrespective of treatment received and to evaluate the treatment effect of belimumab relative to placebo.

In addition to treatment, predictors of PERR at Week 104 indicate that subjects were more likely to achieve renal response if subject had lower baseline proteinuria, were not of black race, had no prior CYC or MMF use, had higher IgM levels, had higher C3 levels, and were female. Region (Asia, Europe, US/Canada, and Americas excluding US/Canada) and renal biopsy class (Class III or Class IV, Class III+V or Class IV+V, Class V only) were among the other effects assessed and neither effect was predictive of PERR response. The results from the model demonstrated that belimumab treatment significantly increased the odds of a Week 104 PERR compared with placebo while controlling for predictive baseline characteristics, yielding an adjusted OR of 1.59 (95% CI 1.05, 2.43; p=0.0306).

Treatment by subgroup interactions were evaluated in the selected multivariable logistic regression model. One significant treatment-by-subgroup interaction (p=0.0039) was observed for baseline proteinuria. In the model including the treatment-by-proteinuria interaction term, treatment remained significant while controlling for the other predictive baseline characteristics.

Multivariable Analysis of CRR at Week 104

A multivariable logistic regression analysis was performed for CRR at Week 104 in the same manner as for PERR.

In addition to treatment, predictors of CRR at Week 104 indicate that subjects were more likely to achieve renal response if subject had lower baseline proteinuria, had no prior CYC or MMF use, and was not positive for anti-dsDNA autoantibodies. Region (Asia, Europe, US/Canada, and Americas excluding US/Canada) and renal biopsy class (Class III or Class IV, Class III+V or Class IV+V, Class V only) were among the other effects assessed and neither effect was predictive of CRR response. The results demonstrated that belimumab treatment significantly increased the odds of a complete renal response at Week 104 compared with placebo yielding an adjusted odds ratio (OR) of 1.73 (95% CI 1.09, 2.75; p=0.0201).

A significant treatment-by-subgroup interaction (p=0.0115) was observed for baseline proteinuria. In the model including the treatment-by-proteinuria interaction term, treatment remained significant for complete renal response at Week 104 while controlling for the other predictive baseline characteristics.

In summary, the multivariable analyses confirmed the treatment effect demonstrated for the Week 104 PERR and CRR, provided support for the greater treatment effect of belimumab vs placebo, and did not indicate region or renal biopsy class as predictive of a response.

### Example 8

### Time to Renal-Related Event or Death

In addition to improvements in achieving and maintaining renal response as confirmed by the PERR and CRR results, belimumab showed superiority in the time to renal-related event or death endpoint (HR=0.51; 95% CI: 0.34, 0.77; p=0.0014) indicating positive effects on prevention of renal function worsening relative to placebo. Renal worsening was the most common event that drove the treatment difference, followed by renal-related treatment failure.

Consistent with the overall population, results favoring belimumab were demonstrated.

### Example 9

### Treating LN Using an IV Dosing Regimen

The Phase 3, randomized, double-blind, placebo-controlled 104-week treatment study evaluated the efficacy and safety of belimumab using IV administration at 10 mg/kg compared to placebo in adult subjects with active lupus nephritis (LN). Key to the dosing regimens in lupus nephritis is maintaining exposure levels whilst decreasing clearance.

All subjects received induction therapy (anytime between −60 days and Day 1) consisting of one of the following standard of care induction/maintenance therapy regimens:

High Dose Corticosteroids (HDCS)+Cyclophosphamide (CYC) for induction therapy followed by Azathioprine (AZA) for maintenance therapy

OR

HDCS+Mycophenolate Mofetil (MMF) for induction therapy followed by MMF for maintenance therapy

Subjects were dosed with Belimumab IV on Days 1 (baseline), 14, 28, and then every 28 days thereafter through 100 weeks with a final evaluation for the double-blind treatment period at 104 weeks. The key efficacy endpoints were selected to reflect long-term renal prognosis.

Although LN is a renal manifestation of SLE, with the same underlying pharmacology in subjects with normal glomerular permeability it is not expected that belimumab (molecular weight 147 kDa) would be cleared via the kidneys due to size restriction of glomerular filtration. However, in patients with LN, inflammation of the glomeruli results in an increase in elimination of intermediate molecular weight proteins and high molecular weight proteins including mAbs and immunoglobulin G (IgG). This increased urinary excretion of proteins (proteinuria) is expected to correlate with increased renal elimination of belimumab resulting in decreased systemic exposure to belimumab.

The primary endpoint was primary efficacy renal response (PERR) at Week 104.

The primary endpoint PERR was defined as estimated Glomerular Filtration Rate (eGFR)≥60 mL/min/1.73 m² or no decrease in eGFR from pre-flare of >20%; and urinary protein:creatinine ratio (uPCR)≤0.7; and not a treatment failure.

The key secondary efficacy endpoints were complete renal response (CRR) at week 104 CRR was defined as eGFR is no more than 10% below the pre-flare value or within normal range; and uPCR<0.5; and not a treatment failure.

Ordinal renal response (ORR) at Week 104—ORR was defined as complete, partial or no response.

Time to renal-related event or death or achieving PERR at a sooner time point such as Week 52.

BEL114054 met the primary and all four key secondary efficacy endpoints, demonstrating superior efficacy with belimumab plus standard therapy compared to placebo plus standard therapy. No new safety issues were identified.

The induction regimens resulted in a rapid reduction in proteinuria over the first 8 to 12 weeks of the study. However, in the Primary Efficacy Renal Response (PERR) in the ≥3 g/g baseline proteinuria subgroup, more subjects on placebo (53.2%) than on belimumab (44.0%) were non-responders due to insufficient renal response. Similar results were noted for the Complete Renal Response (CRR) disposition, where the percentage of non-responders due to insufficient renal response was 67.4% on placebo and 52.8% on belimumab in the ≥3 g/g baseline proteinuria subgroup.

High levels of proteinuria are associated with higher renal elimination of belimumab. In the LN study, at the start of treatment, estimated belimumab clearance (˜334 mL/day) was higher than for adults with SLE (215 mL/day), most likely due to the higher renal component in the clearance of belimumab. Belimumab clearance decreased during the first 24 weeks of the LN study, and from Week 24 onwards, belimumab clearance was similar to that observed in the adult SLE population. Therefore, subjects with higher levels of proteinuria at the start of treatment have lower belimumab systemic exposure compared to subjects with low levels of proteinuria.

Importantly, superiority of belimumab over placebo for the time to renal-related event or death endpoint was demonstrated irrespective of baseline proteinuria level. Prevention of the events associated with increased risk of poor renal survival is an important clinical outcome in all LN patients and especially in those, with advanced and refractory to treatment LN, who are already at increased risk of ESRD.

Although the study was not designed to evaluate long-term effects on renal function, the trends observed in the analyses of eGFR changes over time and proportions of subjects with doubling of serum creatinine/progression to ESRD, suggest beneficial role of belimumab on long-term renal function preservation.

In addition to renal specific measures, benefits of belimumab treatment in the overall SLE management were demonstrated by reduction in SLEDAI-S2K scores and prevention of severe SLE flares. These results are consistent with already established efficacy profile of belimumab in SLE, and further support use of belimumab treatment in LN as a manifestation of a systemic disease.

### Example 10

### PK Simulations for IV Administration

PK simulations were conducted firstly to identify what levels of proteinuria at initiation of treatment may benefit from dose adjustment of belimumab and secondly to evaluate a dosing regimen that would provide belimumab systemic exposure closest to the exposure observed in subjects with low proteinuria.

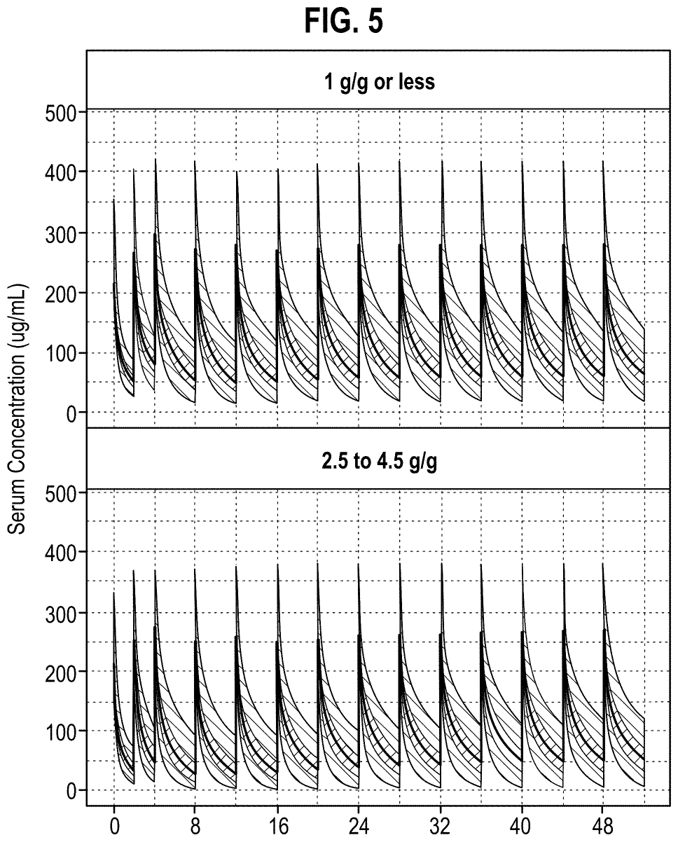

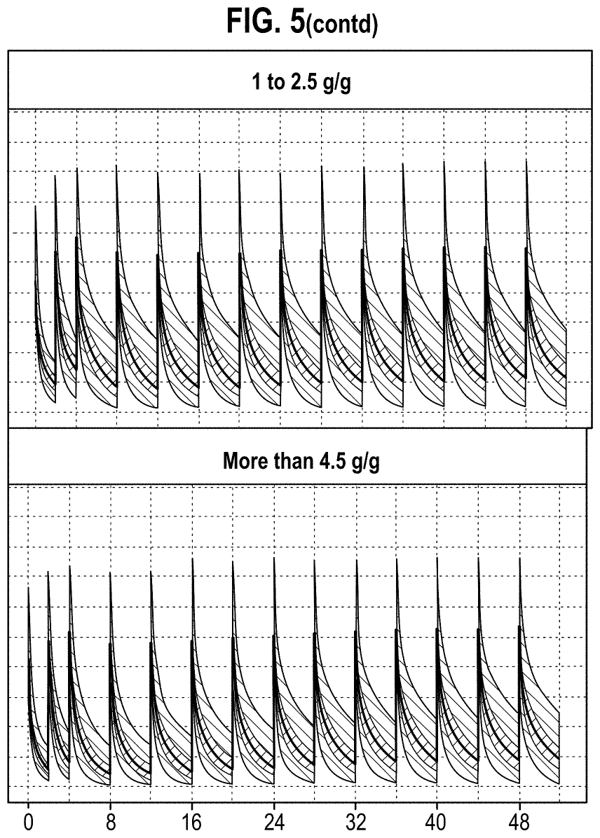

In order to identify what levels of proteinuria may benefit from dose adjustment, the belimumab treated population in the study was divided into 4 groups of approximate quartiles of the baseline proteinuria (uPCR) levels namely <1 g/g, 1 to 2.5 g/g, 2.5 to 4.5 g/g and >4.5 g/g. Predicted concentration time profiles for the SLE approved dosing regimen of IV 10 mg/kg on Day 0, Day 14 (week 2), and Day 28 (week 4) and q4w thereafter, were simulated for each proteinuria group (FIG. 5). The <1 g/g group which is representative of the SLE patient population, was treated as the reference group. For the <1 g/g group, the median average belimumab concentrations (C_{avg}) over the first 12 weeks (107 μg/mL) and the first 24 weeks (105 μg/mL) of treatment were similar to the steady state (SS) belimumab C_{avg} values in subjects with SLE (population median=110 pg/mL and geometric mean=100 μg/mL).

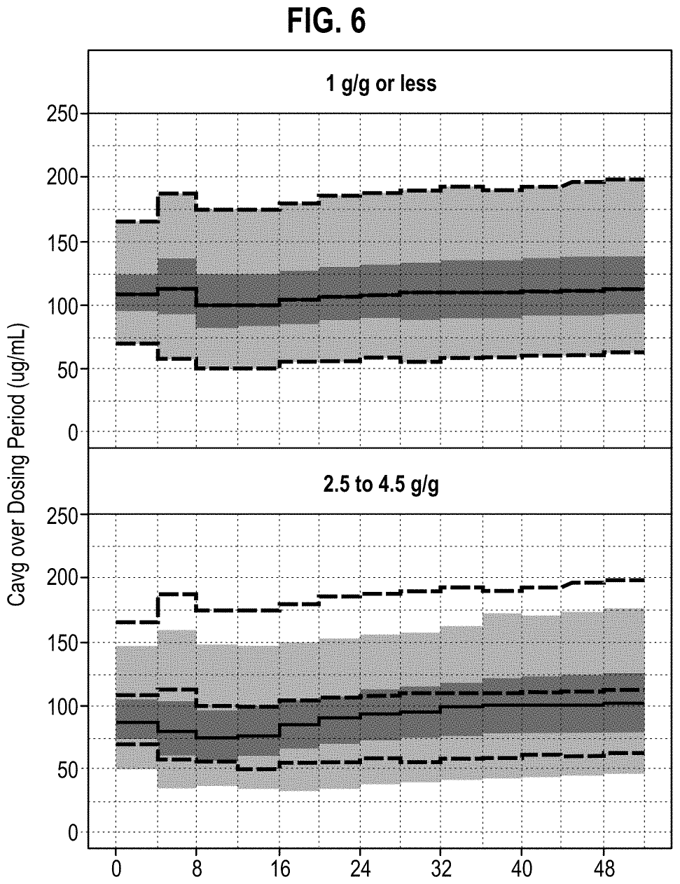

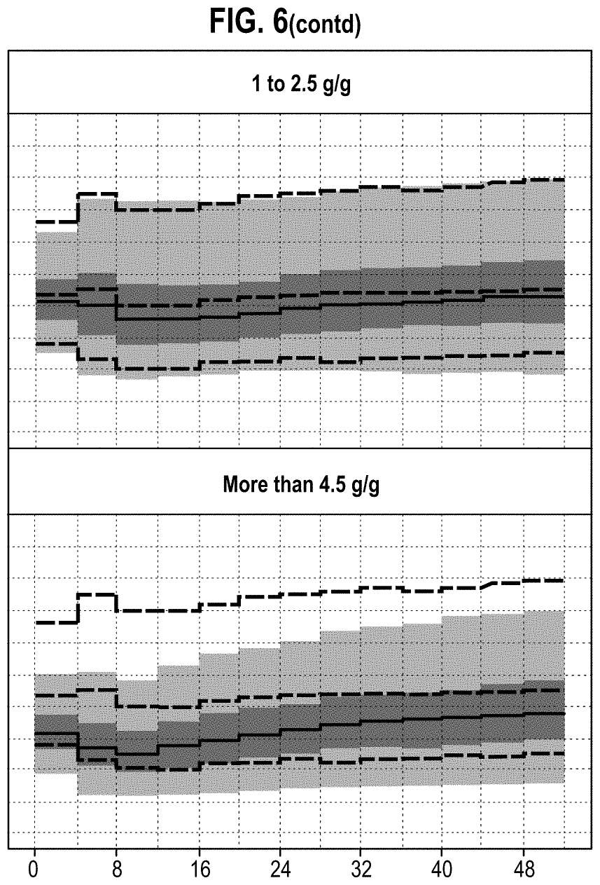

For each simulation, belimumab C_{avg} was calculated over 4 week periods up to week 52. For all proteinuria groups, the C_{avg} tended to be lowest during weeks 4-8, 8-12 and 12-16, compared to weeks 0-4 and weeks 16 onwards. This is most likely due to a balance of loss of the benefit of the Week 2 loading dose, high proteinuria levels at the beginning of treatment and lower belimumab concentration on the approach to steady state (˜12 weeks). During each of the 4 week time periods, the C_{avg} concentrations for the 1 to 2.5 g/g, 2.5 to 4.5 g/g and >4.5 g/g group were lower than the 1 g/g by approximately 10%, 25% and 35-40% respectively (FIG. 6 and Table 2). Since the belimumab C_{avg} for the 1 to 2.5 g/g and 2.5 to 4.5 g/g was within 25% of the 1 g/g group, it was considered that dose adjustment would not be necessary.

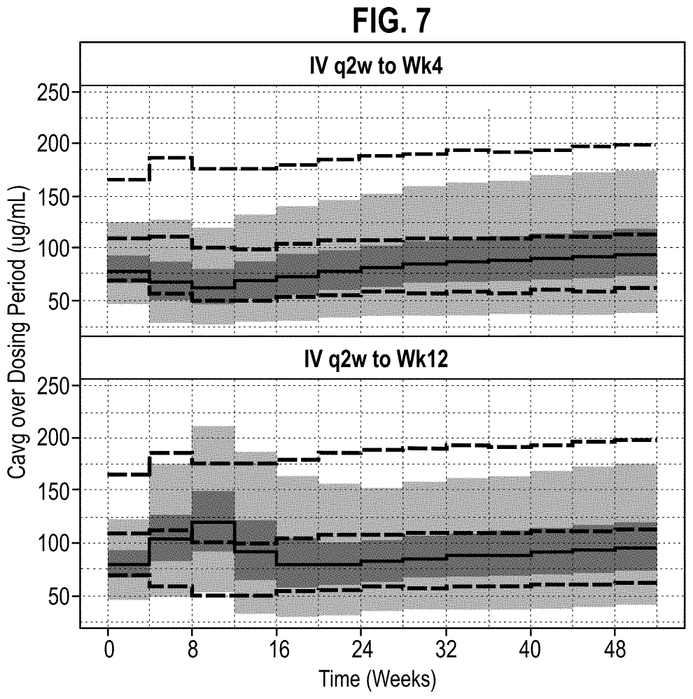

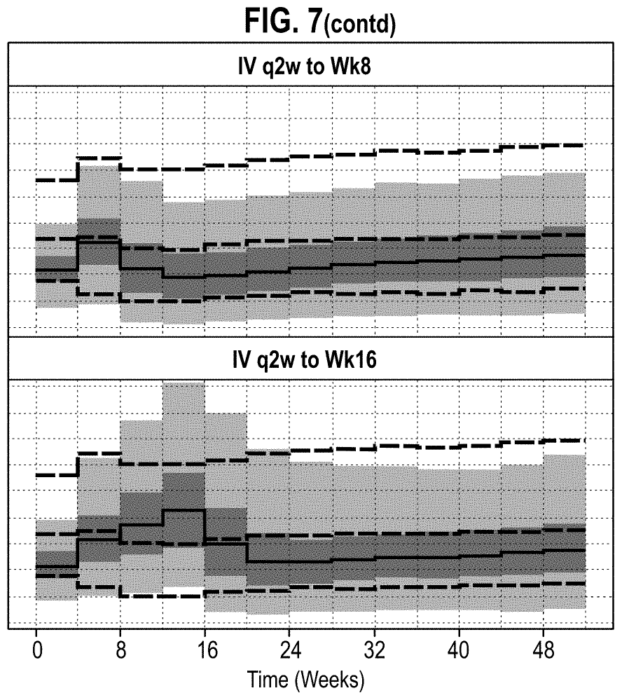

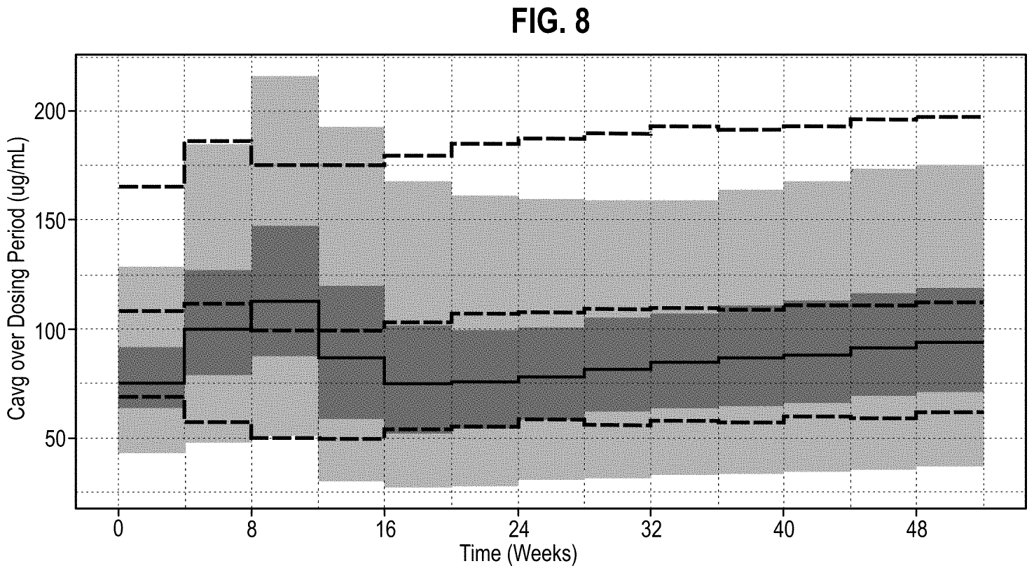

For the >4.5 g/g baseline proteinuria group, q2w dosing to weeks 8, 12 and 16 were simulated each followed by q4w thereafter (FIG. 7 and Table 3). The q2w dosing up to 12 weeks provided the closest match the C_{avg} belimumab values observed in the <1 g/g baseline proteinuria group.

TABLE 2
Predicted Median Belimumab C_{avg} Values Following Administration
of IV 10 mg/kg q2w to Week 4 Followed by 10 mg/kg q4w thereafter
for Different Baseline Proteinuria Subgroups

| | | 1 to 2.5 g/g | | 2.5 to 4.5 g/g | | >4.5 g/g | |
|---|---|---|---|---|---|---|---|
| | <1 g/g | | Ratio | | Ratio | | Ratio |
| | C_{avg} | C_{avg} | vs | C_{avg} | vs | C_{avg} | vs |
| Week | (μg/mL) | (μg/mL) | <1 g/g | (μg/mL) | <1 g/g | (μg/mL) | <1 g/g |
| 0-2 | 89 | 85 | 0.95 | 74 | 0.83 | 68 | 0.76 |
| 2-4 | 128 | 119 | 0.93 | 98 | 0.77 | 87 | 0.68 |
| 4-8 | 112 | 99 | 0.89 | 80 | 0.71 | 67 | 0.60 |
| 8-12 | 100 | 89 | 0.89 | 75 | 0.75 | 62 | 0.62 |
| 12-16 | 99 | 89 | 0.90 | 77 | 0.78 | 68 | 0.69 |
| 16-20 | 103 | 89 | 0.87 | 84 | 0.82 | 73 | 0.71 |
| 20-24 | 107 | 93 | 0.87 | 90 | 0.84 | 77 | 0.72 |
| 24-28 | 108 | 97 | 0.90 | 93 | 0.86 | 81 | 0.75 |
| 28-32 | 109 | 100 | 0.92 | 95 | 0.87 | 84 | 0.77 |
| 0-12 | 107 | 98 | 0.92 | 80 | 0.75 | 69 | 0.65 |

TABLE 3
Predicted Belimumab C_{avg} values for <1 g/g Proteinuria Subgroup Following
Administration of 10 mg/kg q2w to Week 4 Compared to the >4.5 g/g Baseline Proteinuria
Subgroup Following Administration of IV 10 mg/kg q2w to Weeks 4, 8, 12 and 16, with Each
Followed by q4w

| | <1 g/g | >4.5 g/g | | | |
|---|---|---|---|---|---|
| | IV q2w to | IV q2w to | IV q2w to | IV q2w to | IV q2w to |
| | Wk 4 | Wk 4 | Wk 8 | Wk 12 | Wk 16 |
| Weeks | C_{avg} (μg/mL) | | | | |
| 0-2 | 89 | 68 | 69 | 69 | 67 |
| 2-4 | 128 | 87 | 88 | 89 | 86 |
| 4-8 | 112 | 67 | 104 | 104 | 103 |
| 8-12 | 100 | 62 | 78 | 119 | 116 |
| 12-16 | 99 | 68 | 72 | 91 | 130 |
| 16-20 | 103 | 73 | 74 | 79 | 97 |
| 20-24 | 107 | 77 | 78 | 79 | 81 |
| 24-28 | 108 | 81 | 82 | 82 | 81 |
| 28-32 | 109 | 84 | 85 | 85 | 84 |
| 0-12 | 107 | 69 | 87 | 100 | 98 |

### Example 11

### PK Simulations for Subcutaneous Administration

In adults with SLE, the approved SC dose (200 mg q7d) results in a similar belimumab systemic exposure (C_{avg} of 100 μg/mL) and efficacy response as the approved IV dose of 10 mg/kg q4w. Since LN is the renal manifestation of SLE, with the same underlying pharmacology and mode of action for belimumab, similar C_{avg} values for IV and SC dosing are expected to be provide similar efficacy response in LN.

Simulations were conducted to evaluate the impact of a similar duration of SC loading doses but instead of increasing the dose frequency, as with IV administration, the dose of SC was doubled i.e. for <4.5 g/g loading doses of 400 mg q7d on Day 0, week 1, week 2 and week 3 and switch to 200 mg q7d from week 4 (day 28) onwards and for ≥4.5 g/g 400 mg q7d until week 11 and switch to 200 mg q7d from week 12 onwards. These simulations for SC administration confirmed that the predicted belimumab C_{avg} over 4 weekly intervals was similar or lower (first 4 weeks) to the proposed IV dosing regimens.

These simulations confirmed that SC 400 mg loading doses provided belimumab C_{avg} values were comparable to the corresponding IV loading doses to Week 4 in all proteinuria subgroups (Table 4) and to Week 12 for the ≥4.5 g/g baseline proteinuria subgroup (Table 5).

TABLE 4
Predicted Belimumab C_{avg} values Following IV 10 mg/kg
q2w to Week 4 Followed by 10 mg/kg q4w thereafter and
SC Doses of 400 mg q1w to Week 4 Followed by 200 mg q1w

| | <1 g/g | | 1 to 2.5 g/g | | 2.5 to 4.5 g/g | | ≥4.5 g/g | |
|---|---|---|---|---|---|---|---|---|
| | IV | SC | IV | SC | IV | SC | IV | SC |
| Weeks | C_{avg} (μg/mL) | | | | | | | |
| 0-2 | 89 | 52 | 85 | 48 | 74 | 45 | 68 | 41 |
| 2-4 | 128 | 110 | 119 | 97 | 98 | 86 | 87 | 76 |
| 4-8 | 112 | 111 | 99 | 93 | 80 | 80 | 67 | 65 |
| 8-12 | 100 | 100 | 89 | 82 | 75 | 73 | 62 | 59 |
| 12-16 | 99 | 101 | 89 | 83 | 77 | 76 | 68 | 65 |
| 16-20 | 103 | 105 | 89 | 85 | 84 | 80 | 73 | 71 |
| 20-24 | 107 | 106 | 93 | 87 | 90 | 85 | 77 | 75 |
| 24-28 | 108 | 108 | 97 | 90 | 93 | 88 | 81 | 79 |
| 28-32 | 109 | 109 | 100 | 94 | 95 | 90 | 84 | 83 |
| 0-12 | 107 | 97 | 98 | 83 | 80 | 73 | 69 | 61 |

TABLE 5
Predicted Belimumab C_{avg} following IV 10 mg/kg q2w to Week 12
Followed by 10 mg/kg q4w thereafter and SC Loading Doses of
400 mg q1w to Week 12 Followed by 200 mg q1w thereafter
(>4.5 g/g Baseline Proteinuria Subgroup)

| | ≥4.5 g/g | |
|---|---|---|
| Weeks | IV C_{avg} (μg/mL) | SC C_{avg} (μg/mL) |
| 0-2 | 69 | 42 |
| 2-4 | 89 | 78 |
| 4-8 | 104 | 100 |
| 8-12 | 119 | 116 |
| 12-16 | 91 | 96 |
| 16-20 | 79 | 79 |
| 20-24 | 79 | 77 |
| 24-28 | 82 | 80 |
| 28-32 | 85 | 84 |
| 0-12 | 100 | 92 |

Additional systemic safety issues based on the proteinuria dose adjustment recommendations are not anticipated. However, there is the potential for increased incidence of local hypersensitivity reactions when administering 2 subcutaneous injections (400 mg dose). Patients are advised to use a different injection site for each weekly injection and to never give injections into areas where the skin is tender, bruised, red, or hard.

### Example 12

### Simulation and Dose Adjustment

The simulations identified that baseline proteinuria values>4.5 g/g would benefit from dose adjustment. However, from a practical perspective it is considered more appropriate to use a whole integer for uPCR measurement. Therefore, a proteinuria cut off ≥5 g/g at initiation of treatment can be selected for the dose adjustment.

The simulations conducted for the >4.5 g/g group are considered to be reflective of the final ≥5g/g simulations/recommendation since this small difference in proteinuria represents an approximate 2 μg/mL difference in steady-state belimumab C_{avg}. Likewise values of 3.5 or 4.0 g/g may also benefit from dose adjustment in certain patients.

The simulated C_{avg} belimumab concentrations over 4 week time periods for the ≥5 g/g proposed IV q2w dosing up to 12 week and 10 mg/kg q4w thereafter is presented in Error! Reference source not found.8.

Examples of possible dosage regimens are as follows:

IV Low Proteinuria

10 mg/kg on Days 0, 14 and 28, and at 4-week intervals thereafter.

IV High Proteinuria

10 mg/kg at 2-week intervals for the first 12 weeks; then 10 mg/kg at 4-week intervals thereafter.

With persistent proteinuria 10 mg/kg at 2-week intervals may be extended based on physician's judgement.

SC Low Proteinuria

400 mg once weekly (two 200 mg subcutaneous injections) for 4 weeks; then 200 mg once weekly thereafter.

SC High Proteinuria

400 mg once weekly (two 200 mg subcutaneous injections) for 12 weeks; then 200 mg once weekly thereafter.

With persistent proteinuria (500 mg/mmol), 400 mg weekly dosing may be extended based on physician's judgement.

Although the >5 g/g proteinuria level will provide LN patients above the nephrotic range with exposures that are bioequivalent to SLE exposures and without safety implications lower proteinuria levels may also require dose adjustment.

### Example 13

### Bridging from IV Dosing to SC Dosing for LN

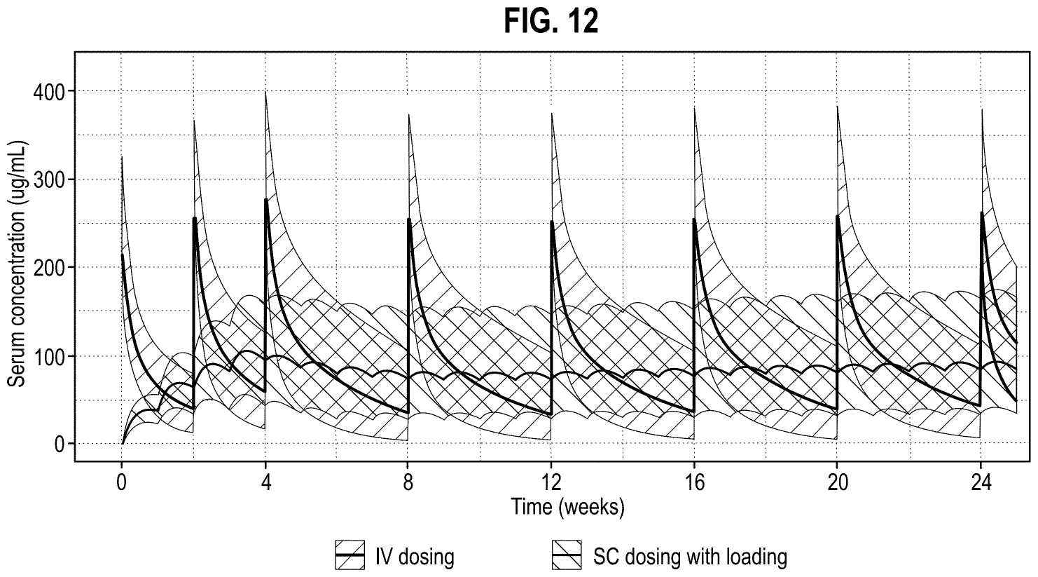

A model-based assessment was conducted to establish bridging from IV to SC in adults with LN. The population PK model developed for the adult LN population was extended to include SC dosing by incorporating the absorption component of the population PK model developed from the adult SLE analysis. It was assumed that bioavailability for SC administration of belimumab was similar in SLE and LN populations. The current approved SC regimen of 200 mg qw (once weekly) does not achieve belimumab steady-state concentrations until approximately week 12 and the exposure to belimumab is lower than the IV dosing regimen during the first 8 weeks of dosing. Given the disease activity in LN and the need for rapid treatment, it is proposed to have initial loading doses of 400 mg qw until week 4, followed by 200 mg qw thereafter. These loading doses provide similar belimumab exposures to those achieved with the IV dosing regimen of 10 mg/kg every 2 weeks (q2w) to week 4, followed by 10 mg/kg q4w thereafter (Table 6 and FIG. 12).

If a patient with lupus nephritis is being transitioned from belimumab intravenous therapy to belimumab subcutaneous therapy, they will be instructed to administer the first subcutaneous dose at least 1 week after the last intravenous dose.

TABLE 6
Predicted Median Belimumab C_{avg} Values Following Administration
of IV 10 mg/kg q2w to Week 4 Followed by 10 mg/kg q4w Thereafter
and SC 400 mg q1w to Week 4 Followed by 200 mg q1w Thereafter
Compared to 200 mg q1w (All Proteinuria Subgroups Combined)

| | IV 10 mg/kg q2w | SC 400 mg q1w | |
|---|---|---|---|
| | to Wk 4 | to Wk 4 | |
| | then q4w | then 200 mg q1w | SC 200 mg q1w |
| Weeks | C_{avg} (μg/mL) | | |
| 0-2 | 80 | 47 | 24 |
| 2-4 | 109 | 92 | 47 |
| 4-8 | 90 | 87 | 62 |
| 8-12 | 81 | 78 | 73 |
| 12-16 | 84 | 81 | 80 |
| 16-20 | 88 | 85 | 85 |
| 20-24 | 92 | 88 | 89 |
| 24-28 | 95 | 91 | 92 |
| 28-32 | 97 | 94 | 94 |
| 0-12 | 89 | 78 | 57 |

The SC dosing regimen has higher predicted belimumab Cmin and lower belimumab Cmax and similar belimumab C_{avg} values compared to the IV dosing regimen (Table 7). This suggests that SC dosing regimen should provide similar efficacy and safety to that observed with IV.

TABLE 7
Predicted Median Belimumab Cₘᵢₙ, C_{avg} and Cₘₐₓ
Values for the Recommended IV and SC Dosing Regimens

| | |
|---|---|
| IV 10 mg/kg q2w | SC 400 mg q1w |
| to Wk 4 then q4w | to Wk 4 200 mg q1w |

| | | | | | |
|---|---|---|---|---|---|
| Cₘᵢₙ | C_{avg} | Cₘₐₓ | Cₘᵢₙ | C_{avg} | Cₘₐₓ |

| | |
|---|---|
| Weeks | (μg/mL) |

| | | | | | | |
|---|---|---|---|---|---|---|
| 0-2 | 0 | 80 | 214 | 0 | 47 | 69 |
| 2-4 | 40 | 109 | 255 | 64 | 92 | 106 |
| 4-8 | 35 | 90 | 275 | 73 | 87 | 101 |
| 8-12 | 32 | 81 | 254 | 69 | 78 | 84 |
| 12-16 | 32 | 84 | 252 | 71 | 81 | 86 |
| 16-20 | 36 | 88 | 255 | 75 | 85 | 90 |
| 20-24 | 39 | 92 | 259 | 79 | 88 | 94 |
| 24-28 | 42 | 95 | 261 | 83 | 91 | 97 |
| 28-32 | 44 | 97 | 264 | 85 | 94 | 100 |

### Example 14

### C_{avg} of Belimumab

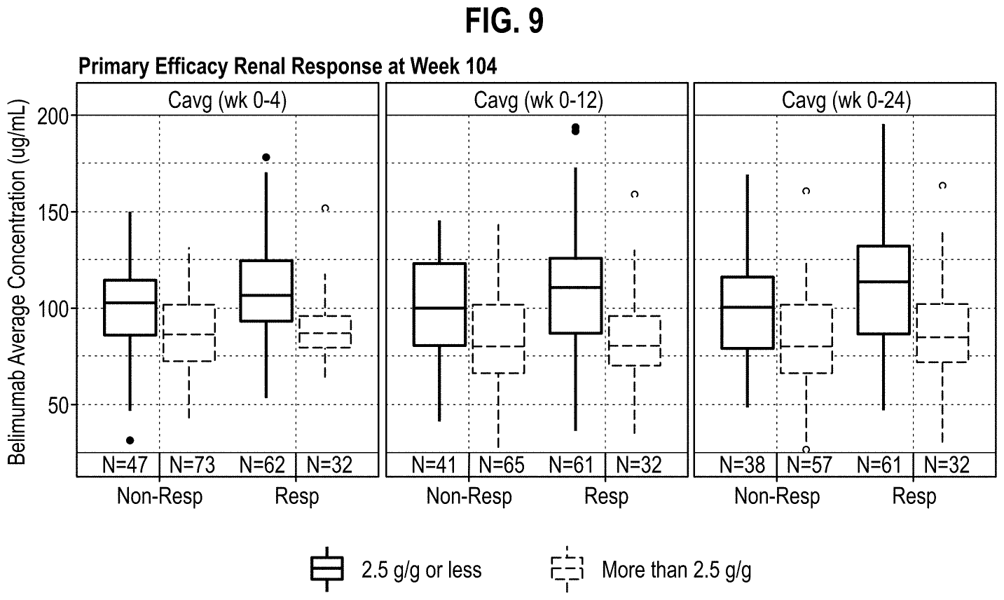

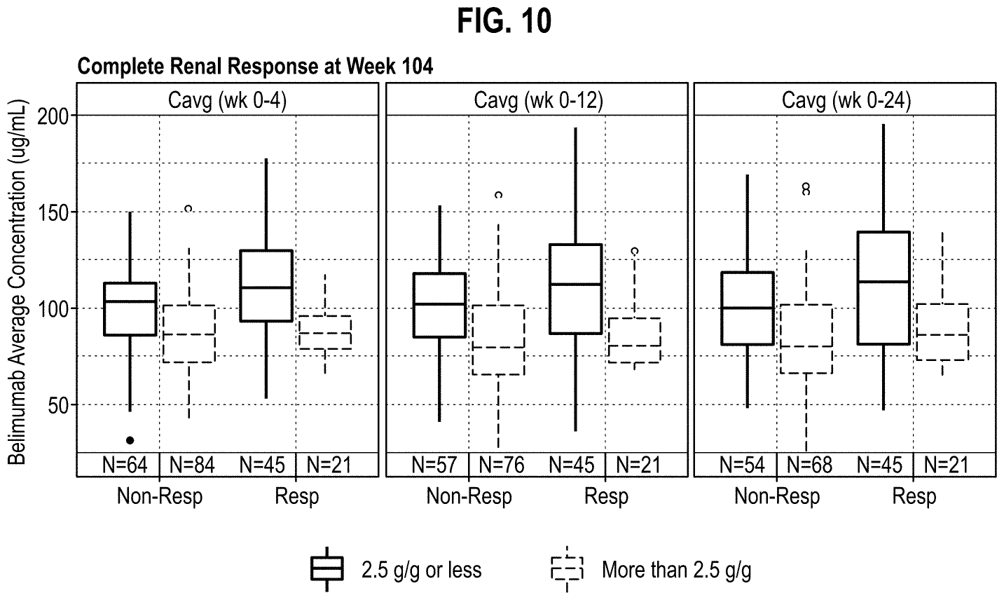

The early time durations selected to determine C_{avg} were weeks 0-4, 0-12 and 0-24. C_{avg} Wk(0-4) was selected as this represents the time period following the first dose of belimumab and the loading dose at Week 2, C_{avg} Wk(0-12) represented the time period with lowest exposure to belimumab due to high proteinuria and switching to the q4w dosing regimen after Week 4. FIG. 9 and FIG. 10 illustrate the C_{avg} in non-responder and responders for PERR and CRR, respectively.

The median C_{avg} Wk(0-4), C_{avg} Wk(0-12) and C_{avg} Wk(0-24) values in the <2.5 g/g baseline proteinuria subgroup are similar to or above C_{avg} values in the SLE population.

For the >2.5 g/g proteinuria subgroup median C_{avg} is lower than the ≤2.5 g/g subgroup. This is likely to be consequence of the higher overall renal disease activity in the >2.5 g/g baseline proteinuria subgroup which results in higher belimumab renal clearance and hence lower belimumab C_{avg}.

Within the >2.5 g/g baseline proteinuria subgroup, the distribution of C_{avg} values is similar for the non-responders and responders for both Week 104 PERR and CRR. In the ≤2.5 g/g subgroup, C_{avg} values appear to be slightly higher in the responders than non-responders for PERR and CRR at Week 104, but the differences were not considered meaningful. Overall, this indicates no association between response at Week 104 and belimumab C_{avg} values in the early weeks of the study.

There was a higher percentage of PERR and CRR responders in the ≤2.5 g/g baseline proteinuria subgroup compared to the >2.5 g/g subgroup for both placebo and belimumab (Table 9). In the ≤2.5 g/g baseline proteinuria subgroup, the percentage of PERR and CRR responders was higher for belimumab than for placebo.

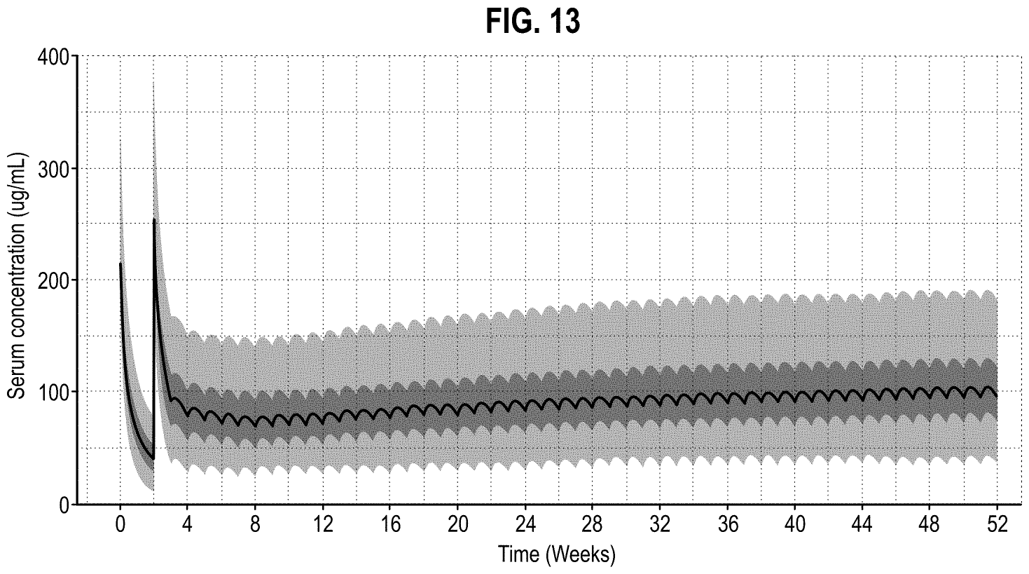

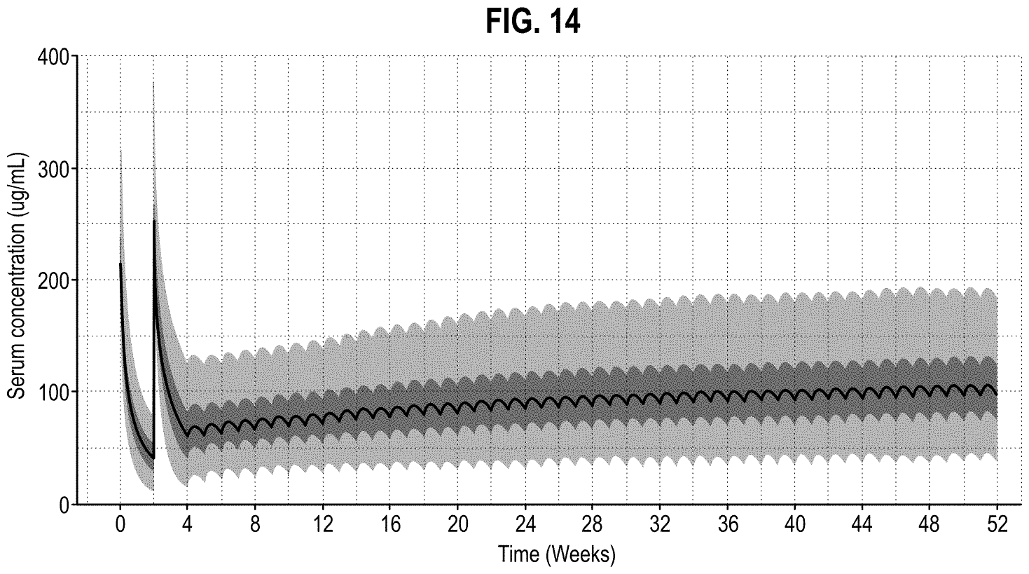

Belimumab concentration time profiles were simulated for switching from IV to SC following the 10 mg/kg q2w loading doses and switched to SC 200 mg q1w either 1 (FIG. 13) or 2 weeks (FIG. 14) after the IV dose at Week 2. At least 2 IV doses of belimumab should be administered before switching to SC.

Switching to SC 1 or 2 weeks after the 2nd IV dose resulted in belimumab C_{avg} values similar to those that achieved with IV 10 mg/kg q4w from week 4 onwards (Table 8). Therefore, if a patient with LN switches from IV to SC they can have the 1st SC dose between 1 and 2 weeks after the last IV dose.

TABLE 8
Predicted Belimumab C_{avg} Values Following Administration of IV
10 mg/kg q2w for 2 Doses Followed by SC 200 mg Q1w at 1 and 2
Weeks After Last IV Dose (All Proteinuria Subgroups
Combined)

| | IV 10 mg/kg q2w for 2 doses and then switch to: | | |
|---|---|---|---|
| | IV 10 mg/kg q4w | SC 200 mg q1w | SC 200 mg q1w |
| | at Wk 4 | at Wk 3 | at Wk 4 |
| Week | C_{avg} (μg/mL) | | |
| 0-2 | 80 | 80 | 80 |
| 2-4 | 109 | 117 | 109 |
| 4-8 | 90 | 79 | 69 |
| 8-12 | 81 | 77 | 75 |
| 12-16 | 84 | 80 | 81 |
| 16-20 | 88 | 85 | 85 |
| 20-24 | 92 | 88 | 89 |
| 24-28 | 95 | 91 | 92 |
| 28-32 | 97 | 93 | 94 |
| 0-12 | 89 | 85 | 81 |

TABLE 9
Percentage of PERR and CRR Responders at Week 104: Stratified by
Baseline Proteinuria

| | PERR | | CRR | |
|---|---|---|---|---|
| | ≤2.5 g/g | >2.5 g/g | ≤2.5 g/g | >2.5 g/g |
| Belimumab (N = 224)^{a} | | | | |
| Number of subjects in | 109 | 115 | 109 | 115 |
| baseline proteinuria | | | | |
| subgroup | | | | |
| Responders, n (%) | 62 (57%) | 32 (28%) | 45 (41%) | 21 (18%) |
| Placebo (N = 224)^{a} | | | | |
| Number of subjects in | 115 | 109 | 115 | 109 |
| baseline proteinuria | | | | |
| subgroup | | | | |
| Responders, n (%) | 41 (36%) | 31 (28%) | 28 (24%) | 16 (15%) |

^{a}Randomized population.

However, replacing exposure with the change in proteinuria over the first 12 weeks of treatment achieved a substantial drop in the objective function, with a higher response rate associated with a larger reduction in proteinuria over the first 12 weeks. This result indicates that the initial change in proteinuria is a better predictor of efficacy than belimumab exposure. The apparent association between exposure and response when dropouts are imputed to NR likely arises because proteinuria is one of the principal determinants of belimumab PK.

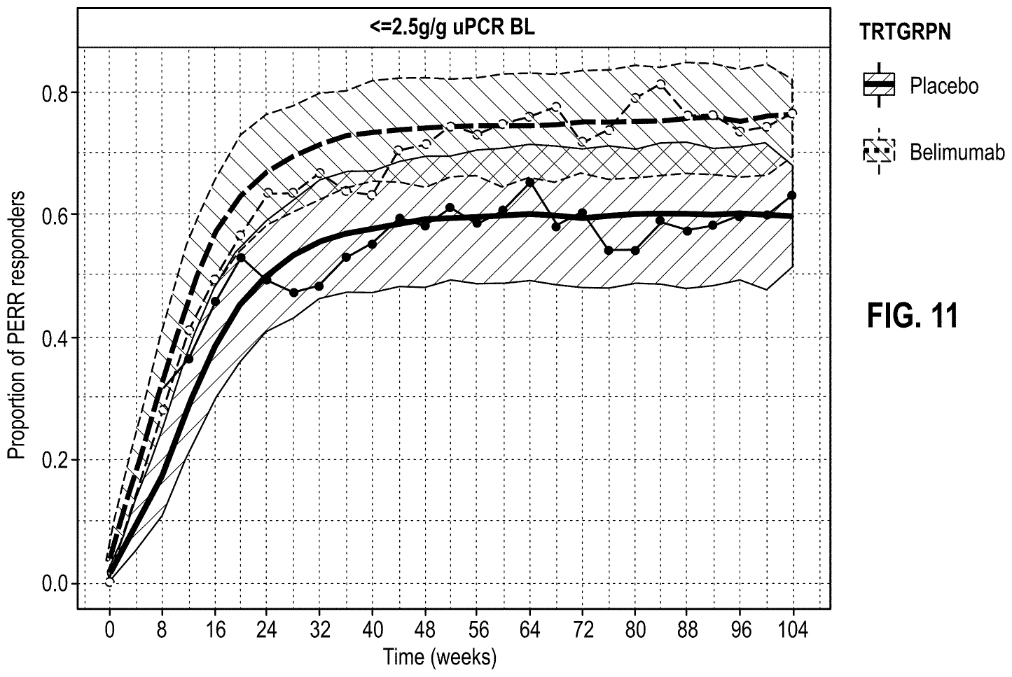

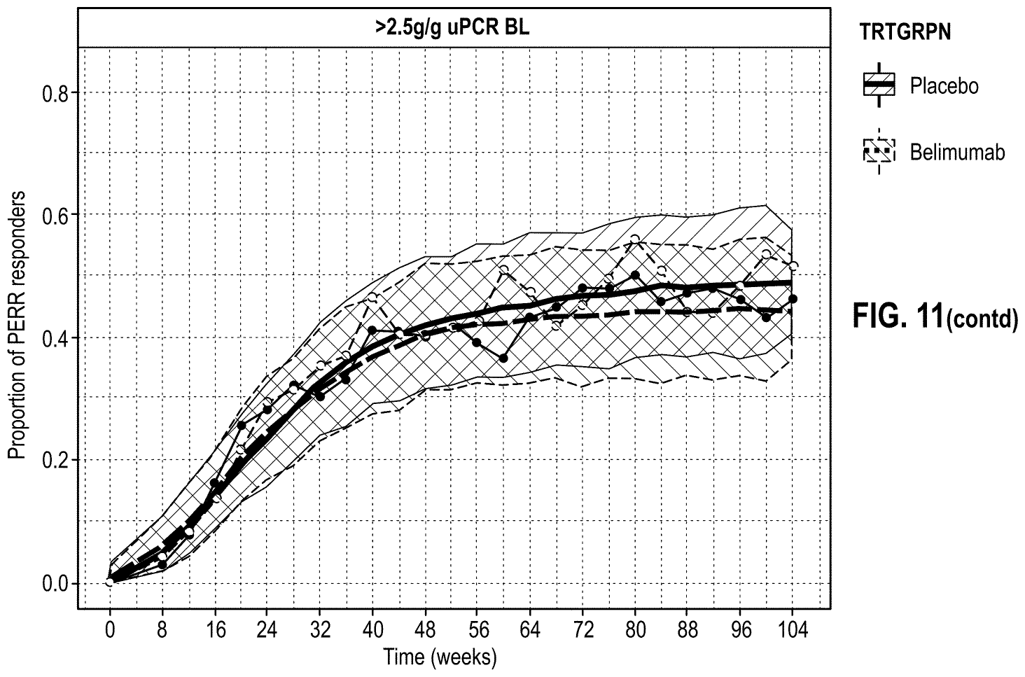

The main conclusions of these analyses should be drawn from the joint dropout/efficacy model rather than the treating off-treatment events as NR. The joint dropout/efficacy model showed that early exposure was not an important covariate of response and was a good description of the data as shown by the visual predictive check (FIG. 11).

### Example 15

### Drug Interaction with Cyclophosphamide

As previously described in the adult SLE marketing applications for the IV and SC formulations of belimumab, the population PK analysis showed no significant effects of a wide range of co-mediations on belimumab PK. With regards to the induction therapies used in BEL114054, the previous population PK for IV and SC SLE showed that MMF had no significant effect on the PK of belimumab.

However, the impact of IV CYC on the PK of belimumab had not been previously evaluated. The population PK analysis for BEL114054 showed that the induction regimen (CYC or MMF) was not a statistically significant covariate of belimumab clearance. Since MMF was previously shown not to have a significant impact on belimumab PK, it can be assumed that CYC would also not have a significant effect on the PK. This is supported by the similarity in the geometric mean belimumab observed pre-dose concentration in the CYC and MMF treated groups (Table 10).

It is therefore proposed that CYC is a co-medication that does not impact the PK of belimumab.

TABLE 10
Observed Pre-Dose Belimumab Concentrations (μg/mL) by
Visit (Double-Blind Phase) (Geometric Mean (CV %)

| | CYC | MMF | Total |
|---|---|---|---|
| | BEL 10 mg/kg | BEL 10 mg/kg | BEL 10 mg/kg |
| Visit | N = 60 | N = 164 | N = 224 |
| Week 24, n | 48 | 134 | 182 |
| | 44.47 (126.60) | 44.60 (90.64) | 44.56 (99.59) |
| Week 52, n | 42 | 128 | 170 |
| | 57.19 (67.50) | 45.64 (76.28) | 48.26 (74.89) |

Note:
Excluded post-″Week 0″ concentrations with a value of ′NQ.′ (non-quantifiable)

### Example 16

### Clinical Pharmacodynamics

The pharmacodynamic responses to belimumab in subjects with LN were consistent with the known mechanism of action of belimumab and previous experience in patients with SLE with the exception of IgG and IgG-based autoantibodies due to urinary excretion of proteins (proteinuria). In contrast to belimumab-treated SLE patients (who experienced significant reductions in IgG), in LN patients, serum IgG levels increased as a result of a rapid decline in urinary excretion of proteins over the first 8 to 12 weeks (which was to a similar extent for belimumab and placebo). At Week 104, the median percent increase from baseline in IgG was 17% for the belimumab and 37% for the placebo group.

A sensitivity analyses to control for changes in urinary IgG loss were performed for anti-double stranded DNA (anti-ds DNA) and C1q antibodies. The results of these analyses demonstrated a notable reduction in anti-dsDNA and anti-C1q antibodies levels relative to the overall level of IgG antibodies in serum for subjects treated with belimumab. Subjects randomized to placebo only showed a modest reduction in the levels of autoantibodies relative to the overall level of IgG antibodies.

Baseline imbalances in absolute values for total CD19+ B cells and B cell subsets were observed in subjects stratified to the different induction/maintenance subgroups but also those randomized within a subgroup; particularly noticeable in CYC/AZA-subgroup. Such baseline imbalances could affect interpretation of the degree of pharmacodynamic effects achieved by each treatment. The relatively lower total CD19+ B cells and subsets thereof observed in CYC-subgroup compared to the MMF-subgroup is likely due to the different mode of action of the induction therapies. It is been reported that CYC mainly reduces naive and memory B cells, whilst sparing transitional B cells and late-stage B cells such as plasmablasts and plasma cells. In contrast, MMF primarily reduces late-stage B cells whilst sparing early stage B cells such as naive and transitional B cells.

Despite the different modes of action of the drugs used in the induction and maintenance regimen, overall pharmacodynamic responses to belimumab in LN patients in the CYC-subgroup and the MMF-subgroup were consistent with the mechanism of action of belimumab.

### Example 17

### Post-Hoc Analysis

BLISS-LN is a landmark study in the field for a number of reasons. It was the first successful Phase 3 randomized controlled trial to show a superior kidney response rate after the addition of a novel immunosuppressive drug to usual therapy. The design of the trial had many unique features including a standard-of-care regimen chosen by each site principal investigator, a very strict glucocorticoid tapering and maintenance schedule, a two-year duration, unique endpoint criteria for the assessment of kidney response, and an evaluation of kidney-related events that are associated with long-term progression to kidney failure. With 10-30% of patients with LN progressing to end-stage kidney disease (ESRD) over time, it is important to assess kidney outcomes directly relevant to long-term kidney health and survival in the BLISS LN trial. The results of these analyses are reported here.

### Results

The study, conducted over 5 years, randomized 448 patients from 107 sites in 21 countries. Eligible patients were ≥18 years of age with autoantibody-positive SLE as per updated American College of Rheumatology classification criteria, and biopsy-confirmed International Society of Nephrology and the Renal Pathology Society 2003-defined Class III, IV, V or mixed LN within 6 months before, or during, screening, showing active lesions or active and chronic lesions. Key exclusion criteria were dialysis or B-cell-targeted therapy (including belimumab) within the preceding year, receipt of CYC induction therapy within 3 months before start of the trial, previous failures of both MMF and CYC induction therapy, and an eGFR of less than 30 ml per minute per 1.73 m² of body surface area. Of the 448 patients studied, 278 (62.3%) completed the treatment through to the scheduled last dose at Week 100 (n=146 [65.5%] and n=132 [59.2%] patients randomized to belimumab and placebo, respectively). Patients who withdrew from the study prematurely were encouraged to attend the remaining scheduled visits despite investigational product discontinuation. Including these individuals, overall, 355 (79.6%) patients completed the study, 186 (52.4%) of whom were in the belimumab arm. The proportion of completers was 83.4% and 75.8% in the belimumab and placebo groups, respectively).

### Response to Belimumab by LN Histologic Class

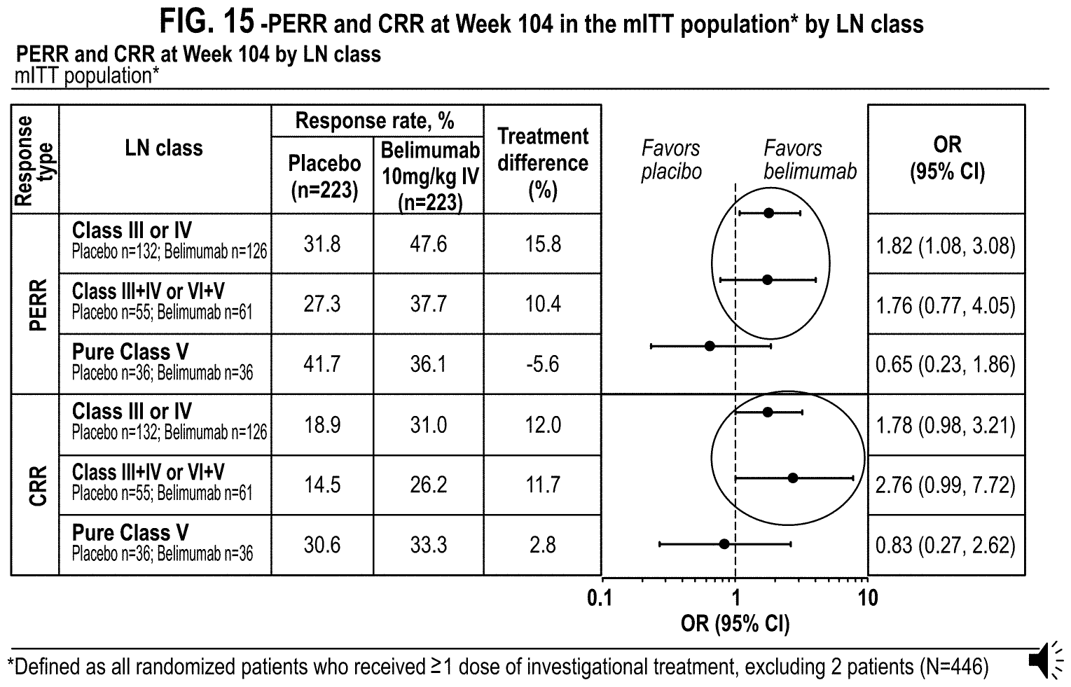

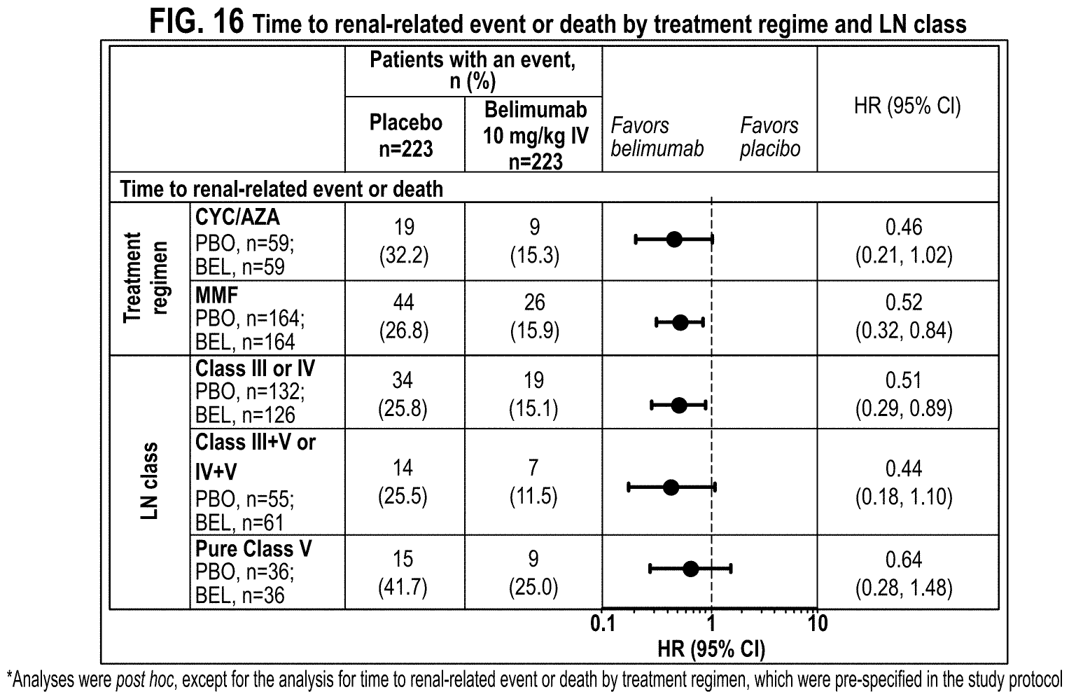

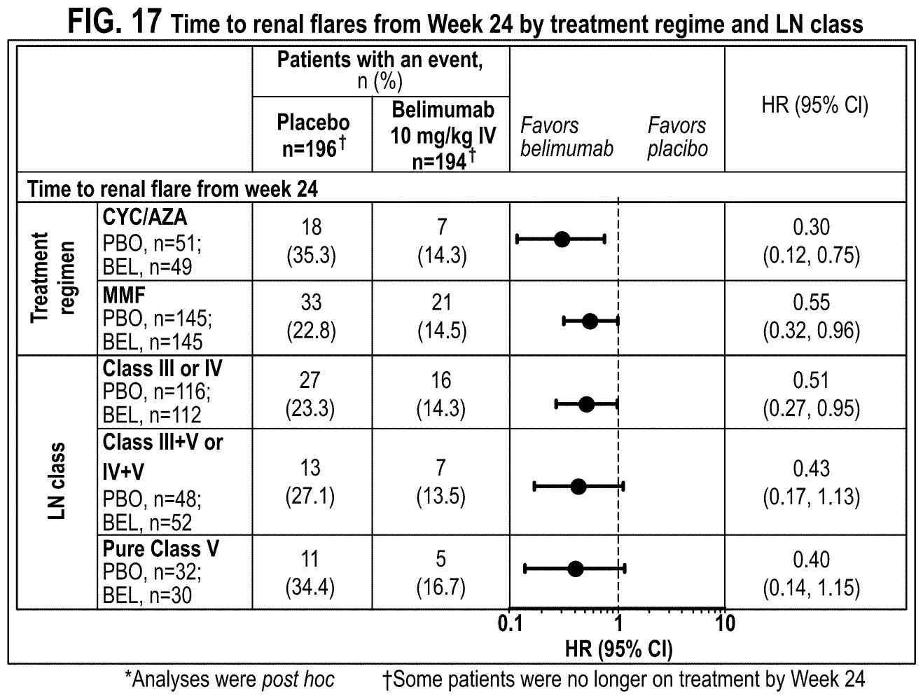

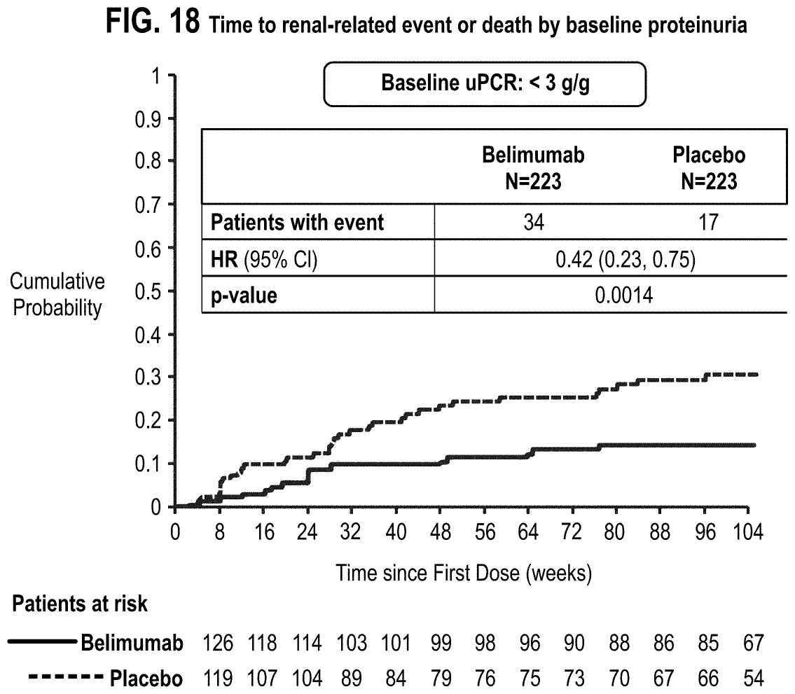

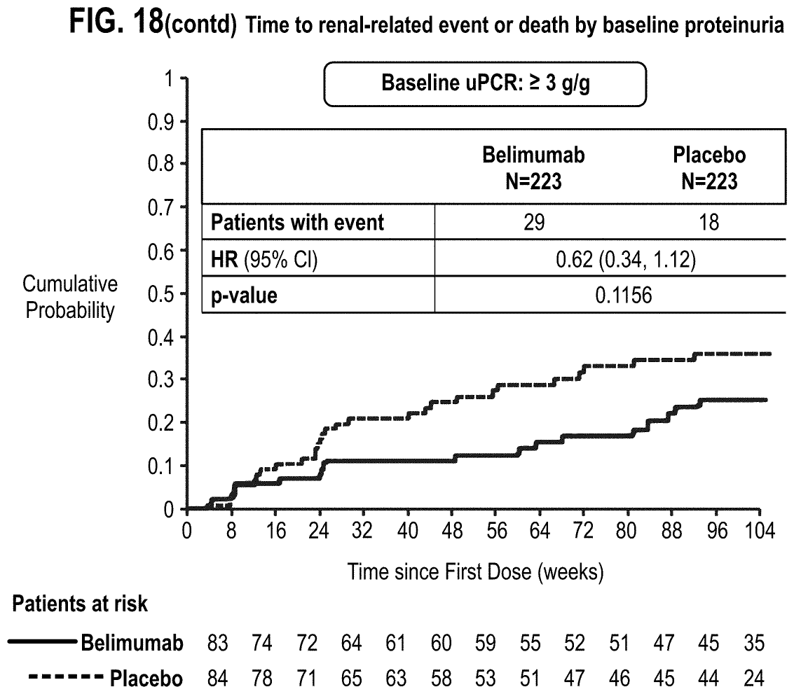

BLISS-LN enrolled patients with both proliferative and membranous forms of LN, although pure Class V LN comprised only 16% of the trial patients. Given the clear differences between these histologic classes, a subgroup analysis was conducted to determine whether response to therapy differed by LN class. As shown in FIG. 15 the overall primary efficacy renal response (PERR) and complete renal response (CRR) outcomes favoring belimumab over placebo were driven mainly by patients with a proliferative histologic component (Classes III and IV with or without Class V). There was no treatment difference observed for PERR or CRR in patients with pure Class V LN.

### Effect of Belimumab on Time to Renal-Related Events

Time to a renal-related event or death was a key secondary outcome of the BLISS-LN trial, and as noted in the trial's primary analysis, this risk was significantly reduced in belimumab-treated patients. Renal-related events were mainly due to increased proteinuria, decreased kidney function, or both. When stratified by induction regimen used (mycophenolate mofetil [MMF] or cyclophosphamide [CYC]/azathioprine [AZA]) or LN class, the risk of a renal-related event was reduced by belimumab independent of induction treatment and independent of LN class (Error! Reference source not found.16).

Because the BLISS-LN study was done over 2 years, there was opportunity to evaluate the effects of belimumab on LN flare. LN flares were assessed after trial week 24, and the Week 24 clinical data (proteinuria and kidney function) served as the baseline for flare adjudication. The results of the time to renal flare from Week 24 post hoc analyses were similar to the results of the time to a renal-related event or death endpoint. Overall, during the study, treatment with belimumab reduced the risk of experiencing an LN flare. Interestingly, the LN flare risk reduction in CYC/AZA-treated patients was greater than in MMF-treated patients (Error! Reference source not found. 17). Belimumab also attenuated risk for all LN classes when time to first LN flare after Week 24 was stratified by class, consistent with the overall population results. Time to renal flare was also examined in those patients who achieved either a PERR or a uPCR below 0.5 at Week 24, and for each subgroup there were fewer flares in patients who received belimumab.

TABLE 11

| | Baseline uPCR | | Baseline uPCR | |
|---|---|---|---|---|
| | <3 g/g | | ≥3 g/g | |
| | | Belimumab | | Belimumab |
| | Placebo | 10 mg/kg | Placebo | 10 mg/kg |
| Subgroup | (n = 131) | (n = 132) | (n = 92) | (n = 91) |
| Total events^{†}, n (%) | 34 (26.0) | 17 (12.9) | 29 (31.5) | 18 (19.8) |
| Death | 1 (0.8) | 1 (0.8) | 1 (1.1) | 0 |
| Progression to ESRD | 1 (0.8) | 0 | 0 | 0 |
| Doubling of creatinine | 0 | 1 (0.8) | 1 (1.1) | 0 |
| from baseline | | | | |
| Renal worsening^{‡} | 23 (17.6) | 9 (6.8) | 16 (17.4) | 8 (8.8) |
| Renal treatment failure^{§} | 9 (6.9) | 6 (4.5) | 11 (12.0) | 10 (11.0) |

### Effect of Belimumab on Kidney Function

The overall goal of LN treatment is to preserve kidney function. All LN trials to date have used varied definitions of kidney response, generally 6-12 months after trial entry as a surrogate of good long-term kidney health. All response definitions use a combination of proteinuria, kidney function (serum creatinine concentration or estimated glomerular filtration rate [eGFR]), and often urine sediment. The BLISS-LN trial, because of its duration, also afforded the opportunity to examine changes in eGFR over two years in patients treated with and without belimumab. The slope of eGFR change was assessed between Week 24 and Week 104 in patients while they remained on treatment and while they remained in the study, even if treatment may have been withdrawn (Table 22). All patients showed a decline in eGFR over the course of the study (that is a negative eGFR slope); however, in both patient groups, on-treatment or on-study, the decline in eGFR over the duration of the trial was less in belimumab-treated patients than placebo-treated patients (Table 22).

TABLE 22
Change in kidney function in the mITT population between Week 24 and 104

| | | | On-Study | |
|---|---|---|---|---|
| | | | (some no longer on study | |
| | On-treatment | | treatment) | |
| | | Belimumab | | Belimumab |
| | Placebo | 10 mg/kg IV | Placebo | 10 mg/kg IV |
| | (n = 223) | (n = 223) | (n = 223) | (n = 223) |
| Patients at any visit, n | 198 | 196 | 198 | 196 |
| Patients at Week 104, n | 128 | 140 | 163 | 173 |
| Mean eGFR (SE) at Week 24^{†} | 106.6 (2.49) | 109.4 (2.36) | 106.8 (2.55) | 109.5 (2.39) |
| eGFR slope (ml/min/1.73 m²/year) (SE)^{†} | −3.18 (1.10) | −0.99 (0.77) | −5.72 (1.47) | −2.12 (0.97) |
| eGFR slope difference vs placebo (SE)^{†} | | 2.19 (1.34) | | 3.61 (1.76) |
| 95% Cl | — | (−0.45, 4.84) | — | 0.15, 7.06 |
| p-value | | 0.1041 | | 0.0407 |

The effect of belimumab on kidney function was also assessed as time to a decline in eGFR of 30% or 40%, two GFR milestones considered as predictors of future kidney insufficiency or failure. For this analysis, patients were censored either at belimumab/placebo treatment discontinuation, or at trial withdrawal. For both censored patient groups more patients receiving placebo had a 30% or 40% decline in eGFR than those treated with belimumab (Table 1). In both patient groups belimumab significantly attenuated the time to first decline in eGFR by 30% or 40% with hazard ratios between 0.35 and 0.52 (Table 1).

TABLE 22
Time to 30% and 40% decline in eGFR

| | Censored at withdrawal or | | | |
|---|---|---|---|---|
| | treatment discontinuation | | Censored at withdrawal | |
| | | Belimumab | | Belimumab |
| | Placebo | 10 mg/kg IV | Placebo | 10 mg/kg IV |
| | (n = 223) | (n = 223) | (n = 223) | (n = 223) |
| Patients with 30% decrease in eGFR | 12.6% | 6.7% | 17.0% | 8.5% |
| 30% decrease in eGFR, HR | | 0.52 | | 0.47 |
| 95% Cl | | (0.28, 0.98) | | (0.27, 0.83) |
| p-value | | 0.0429 | | 0.0084 |
| Patients with 40% decrease in eGFR | 6.7% | 2.7% | 11.7% | 4.5% |
| 40% decrease in Egrf, HR | | 0.38 | | 0.35 |
| 95% Cl | | (0.15, 0.98) | | (0.17, 0.74) |
| p-value | | 0.0457 | | 0.0056 |

### Effect of Baseline Proteinuria on the Efficacy of Belimumab

Because the level of proteinuria at flare may affect the rapidity and completeness of the renal response in LN, patients were divided by level of baseline proteinuria at trial entry into those with <3 g/d (n=263) and those with ≥3 g/d (n=183) and PERR and CRR were evaluated. As shown in Table, the effects of belimumab favoring PERR and CRR were driven by patients with baseline proteinuria<3 g/d. There were no treatment differences in patients with high baseline proteinuria. In contrast, when time to a renal-related event or death was stratified by level of baseline proteinuria, belimumab was effective in reducing risk in patients with low and high baseline proteinuria (Error! Reference source not found.18). The events contributing to the renal-related event outcomes are described in Table 15.

We also examined the effect of baseline proteinuria on eGFR slope and the time to a 30% or 40% decline in eGFR were reanalyzed after segregation on baseline level of proteinuria. The risk of reaching a 30% or 40% decline in eGFR was reduced in patients treated with belimumab regardless of the level of baseline proteinuria (Table 16). Belimumab also attenuated the rate of eGFR decline at both levels of proteinuria in patients who were on study treatment at Week 104.In contrast, eGFR decline was steeper in placebo patients who remained in the study but were off treatment, and there was no observed benefit of belimumab in patients who were off study treatment who had a high level of baseline proteinuria.

TABLE 15
Renal-related events or death by proteinuria category

| | Baseline uPCR | | Baseline uPCR | |
|---|---|---|---|---|
| | <3 g/g | | ≥23 g/g | |
| | | Belimumab | | Belimumab |
| | Placebo | 10 mg/kg | Placebo | 10 mg/kg |
| Subgroup | (n = 131) | (n = 132) | (n = 92) | (n = 91) |
| Total events^{†}, n (%) | 34 (26.0) | 17 (12.9) | 29 (31.5) | 18 (19.8) |
| Death | 1 (0.8) | 1 (0.8) | 1 (11) | 0 |
| Progression to ESRD | 1 (0.8) | 0 | 0 | 0 |
| Doubling of creatinine | 0 | 1 (0.8) | 1 (1.1) | 0 |
| from baseline | | | | |
| Renal worsening^{‡} | 23 (17.6) | 9 (6.8) | 16 (17.4) | 8 (8.8) |
| Renal treatment failure^{§} | 9 (6.9) | 6 (4.5) | 11 (12.0) | 10 (11.0) |

TABLE 16
Time to Confirmed 30% and 40% Decrease in eGFR from baseline
by baseline uPCR.

| IPD/WD = Censor | | Treatment Policy | |
|---|---|---|---|
| Belimumab | | Belimumab | |
| 10 ma/kg | Placebo | 10 mg/kg | Placebo |
| (n = 223) | (n = 223) | (n = 223) | (n = 223) |

Baseline uPCR <3 g/g

| | | | | |
|---|---|---|---|---|
| n | 132 | 131 | 132 | 131 |
| Patients with confirmed 30% | 6 (4.5) | 14 (10.7) | 8 (6.1) | 19 (14.5) |
| decrease in eGFR, n (%) | | | | |
| 30% decrease in eGFR Hazard | 0.35 | | 0.29 | |
| Ratio ^{a} | (0.13, 0.91) | | (0.12, 0.68) | |
| 95% Cl ^{a} | 0.0320 | | 0.0045 | |
| P-value ^{a} | | | | |
| n | 132 | 131 | 132 | 131 |
| Patients with confirmed 40% | 3 (2.3) | 9 (6.9) | 5 (3.8) | 15 (11.5) |
| decrease in eGFR, n (%) | | | | |
| 40% decrease in eGFR Hazard | Number of | | 0.20 | |
| Ratio* | events too | | (0.07, 0.60) | |
| 95% Cl* | low to | | 0.0041 | |
| P-value* | estimate | | | |

Baseline uPCR ≥3 g/g

| | | | | |
|---|---|---|---|---|
| n | 91 | 92 | 91 | 92 |
| Patients with confirmed 30% | 9 (9.9) | 14 (15.2) | 11 (12.1) | 19 (20.7) |
| decrease in eGFR, n (%) | | | | |
| 30% decrease in eGFR Hazard | 0.76 | | 0.63 | |
| Ratio* | (0.33, 1.78) | | (0.30, 1.33) | |
| 95% Cl* | 0.5309 | | 0.2273 | |
| P-value* | | | | |
| n | 91 | 92 | 91 | 92 |
| Patients with confirmed 40% | 3 (3.2) | 6 (6.5) | 5 (5.5) | 11 (12.0) |
| decrease in eGFR, n (%) | | | | |
| 40% decrease in eGFR Hazard | Number of | | 0.45 | |
| Ratio* | events too | | (0.15, 1.30) | |
| 95% Cl* | low to | | 0.1395 | |
| P-value* | estimate | | | |

Based on this secondary post hoc analysis of BLISS-LN, if success in the management of LN depends only on whether a PERR or CRR is achieved at the end of one or two years of treatment, belimumab should be considered as an add-on therapy for proliferative LN patients with modest proteinuria who are being treated with MMF. But the key goal of LN management is long-term preservation of kidney function, and not simply meeting a single, arbitrary proteinuria and eGFR endpoint. As shown here, the addition of belimumab to background therapy attenuated the decline in eGFR and decreased the number of patients reaching a 30-40% fall in eGFR. This apparent preservation of kidney function was seen across the two most commonly used LN background therapies, across LN histologic classes, and across baseline levels of proteinuria, if patients remained on treatment. These data shows belimumab can be used in any LN patient to maintain kidney survival.

The ability of belimumab to preserve kidney function is most readily explained by the observation that belimumab-treated patients had less renal worsening and few LN flares than placebo-treated patients. Progressive loss of kidney function in LN is due to the accumulation of chronic injury to the renal parenchyma. This occurs during every LN flare as inflammatory lesions heal with scar formation. Additionally, even in the absence of clinically defined LN flares, patients with LN may suffer from ongoing, subclinical inflammation that progressively expands chronic kidney injury. This generally manifests as worsening proteinuria and/or eGFR over time. The absence of disease quiescence in LN facilitates progression of chronic kidney injury toward ESRD. As shown here, belimumab prevented renal worsening and overt LN flares independent of LN background therapy, LN histologic class, or baseline level of proteinuria.

In addition to providing kidney protection by maintaining LN quiescence, belimumab may also help attenuate renal fibrosis directly. A small study of patients with systemic sclerosis examined transcript expression in skin biopsies from patients who improved after belimumab was added to their therapeutic regimen. The pathways down-regulated in belimumab-treated patients were enriched for matrix expression and inflammation. In a murine model of pulmonary fibrosis induced by bleomycin, lung injury was attenuated by blocking or knocking out B Lymphocyte Stimulator (BLyS). Pulmonary fibrosis was IL-17A-dependent, and IL-17A expression was enhanced by exogenous BLyS. Such mechanisms may also be relevant in the LN kidney, and BLyS inhibition by belimumab could therefore have a more direct role in reducing chronic kidney damage.

Differences in the effect of belimumab on renal response (PERR and CRR) and its effects on preservation of kidney function, especially when patients are segregated into subgroups of induction therapy, LN class, and baseline proteinuria are not always clear. The answer to this apparent disassociation may lie in the data used to establish PERR as the primary endpoint of BLISS-LN. Several studies demonstrated good long-term kidney outcomes even if patients do not dip below the 500 mg/d proteinuria cutoff used in most definitions of CRR. These studies showed that achieving proteinuria of 700-800 mg/d after a year of treatment was sufficient to ensure good long-term kidney function. However in two of these studies, the proteinuria cutoff below 700-800 mg/d, despite having a good positive predictive value for preservation of kidney function, had a low negative predictive value. That is, patients who achieved this level of proteinuria after a year of treatment did well, but so did many patients who did not reach this cutoff. While it is acknowledged that control or remission of proteinuria is critical for good long-term outcomes in all glomerular diseases, it is not clear that this must be within a fixed interval of time after the start of treatment. In addition, the known discordance between clinical renal response and histologic renal response may also have a role. Studies have demonstrated that resolution of immune injury to the kidneys is not always reflected in resolution of clinical markers such as proteinuria. Addressing this issue would require a kidney biopsy to evaluate patients who have been treated with belimumab but have not reached PERR or CRR to understand whether histologic activity has resolved.

Belimumab did not increase PERR or CRR for patients with Class V LN. This may inform the design of future LN trials. While the histology of membranous LN is very different than that of proliferative LN, and this may reflect at least some differences in the pathogenesis of Class V compared with Classes III and IV, most trials have included proliferative and membranous LN patients. This was done for the practical need of adequate trial enrollment, and because non-specific immunosuppressants (MMF, CYC) have been used successfully to treat all LN classes. As more targeted immunosuppressants are evaluated it may be reasonable to enrich trial enrollment with patients that may be more likely to benefit from the novel agents. Of course, such enrichment will be enhanced as more insight into the pathogenic mechanisms of LN phenotypes is obtained.

LN flares were defined as impaired kidney function defined as a reproducible eGFR decrease of >20% from Week 24, accompanied by proteinuria (>1 g) and/or cellular casts (red-cell, white-cell, or both), or as an increase in proteinuria compared with Week 24. The latter was defined as an increase in urinary protein:creatinine ratio [uPCR] of >1 if Week 24 uPCR (uPCR₂₄) was <0.2 g/g, to >2.0 if uPCR₂₄ was 0.2-1 g/g, or as a doubling in uPCR from Week 24 if uPCR₂₄ was >1 g/g. Of note, the analysis of LN flares included patients, who achieved PERR at Week 24 and patients with uPCR₂₄≤0.5. For the primary analysis, treatment failure due to kidney disease-related intake of prohibited medications was considered a LN flare. Of note, these treatment failure-defined flares did not factor in the analysis of time to LN flare from Week 24. Instead, this analysis was based solely on eGFR and/or uPCR criteria.

Time to confirmed eGFR decline of 30% and 40% were measured from study baseline, i.e., Day 0, and analyzed for two different datasets. The first dataset included all eGFR measurements obtained while patients received treatment (‘on treatment’), in which data from patients who discontinued belimumab or placebo or withdrew from the trial was censored. The second analysis included all eGFR measurements obtained while patients remained enrolled in the study, even if they prematurely discontinued belimumab or placebo (‘on study’), with withdrawals being censored.

The annual rate of eGFR decline was evaluated as a chronic slope from Week 24 to account for acute effects of induction therapy early in the study on renal function. Like the time to confirmed eGFR decline, this analysis was performed for both the ‘on-treatment’ and the ‘on-study’ population.

Statistical Analysis

Efficacy endpoints were analyzed in the modified intention-to-treat population (mITT) that included all randomized patients who received at least one dose of belimumab or placebo (n=223 for both belimumab and placebo group). Two patients in the total population (n=446) were excluded from the mITT population due to compliance issues at the respective research site.

Statistical models controlled for race or ethnicity, induction regimen, as well as eGFR and uPCR at baseline. In the analysis of PERR and CRR, study product (belimumab or placebo) discontinuation, study withdrawal, or treatment failure were inputted as ‘no response’.

The endpoints of the PERR and CRR were analyzed with logistic regression. The time to a renal-related event or death was analyzed in a Cox proportional-hazards regression model in which data from patients who discontinued the study product (belimumab or placebo), withdrew from the study before occurrence of a renal-related event or death, or had a treatment failure unrelated to a kidney event was censored.

The annual rate of eGFR decline was estimated from a linear mixed model consisting of treatment group (belimumab vs placebo), analysis visit (study week), and their interaction, and random intercept and slope at the patient level. Covariance structure for random intercept and slope was unstructured and heterogeneous for treatment groups.

Belimumab was particularly effective in inducing PERR and CRR in patients with baseline proteinuria of 3 g/d or less. In general, however, patients with proteinuria of 3 g/d or more have histologically more active disease, and it is possible that simply more time is needed to resolve intra-renal inflammation.

In conclusion, these data suggest that belimumab added to standard-of-care may be effective in preserving kidney function in the long-term for all patients with LN. This likely occurs through several pathways, however, preventing renal worsening and LN flares is undoubtedly important.

It is to be understood that the invention is not limited to the aspects or embodiments illustrated hereinabove and the right is reserved to the illustrated aspects or embodiments and all modifications coming within the scope of the following claims.

SEQUENCE LISTING

SEQ ID NO: 1: Belimumab CDRH1
GGTFNNNAIN
SEQ ID NO: 2: Belimumab CDRH2
GIIPMFGTAKYSQNFQG
SEQ ID NO: 3: Belimumab CDRH3
SRDLLLFPHHALSP
SEQ ID NO: 4: Belimumab CDRL1
QGDSLRSYYAS
SEQ ID NO: 5: Belimumab CDRL2
GKNNRPS
SEQ ID NO: 6: Belimumab CDRL3
SSRDSSGNHWV
SEQ ID NO: 7: Belimumab VH
QVQLQQSGAEVKKPGSSVRVSCKASGGTFNNNAINWVRQAPGQGLEWMG
GIIPMFGTAKYSQNFQGRVAITADESTGTASMELSSLRSEDTAVYYCAR
SRDLLLFPHHALSPWGRGTMVTVSS
SEQ ID NO: 8: Belimumab VL
SSELTQDPAVSVALGQTVRVTCQGDSLRSYYASWYQQKPGQAPVLVIYG
KNNRPSGIPDRFSGSSSGNTASLTITGAQAEDEADYYCSSRDSSGNHWV
FGGGTELTVLG
SEQ ID NO: 9: Belimumab heavy chain
QVQLQQSGAEVKKPGSSVRVSCKASGGTFNNNAINWVRQAPGQGLEWMG
GIIPMFGTAKYSQNFQGRVAITADESTGTASMELSSLRSEDTAVYYCAR
SRDLLLFPHHALSPWGRGTMVTVSSASTKGPSVFPLAPSSKSTSGGTAA
LGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPS
VFLFPPKPKDTLMISRTPEVTCVVVDVSHEDPEVKFNWYVDGVEVHNAK
TKPREEQYNSTYRWVSVLTVLHQDWLNGKEYKCKVSNKALPAPIEKTIS
KAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNH
YTQKSLSLSPGK
SEQ ID NO: 10: Belimumab light chain
SSELTQDPAVSVALGQTVRVTCQGDSLRSYYASWYQQKPGQAPVLVIYG
KNNRPSGIPDRFSGSSSGNTASLTITGAQAEDEADYYCSSRDSSGNHWV
FGGGTELTVLGQPKAAPSVTLFPPSSEELQANKATLVCLISDFYPGAVT
VAWKADSSPVKAGVETTTPSKQSNNKYAASSYLSLTPEQWKSHRSYSCQ
VTHEGSIVEKTYAPTECS

1-51. (canceled)

52. A method of treating lupus nephritis in a human patient in need thereof, comprising administering to the patient a therapeutically effective amount of an anti-BLyS antibody, wherein the patient has active lupus nephritis.

53. The method according to claim 52, wherein the antibody is belimumab.

54. The method according to claim 53, wherein the antibody is administered intravenously.

55. The method according to claim 54, wherein the antibody is administered to the patient at a dose of 10 mg/kg.

56. The method according to claim 55, wherein the antibody is administered every 2 weeks.

57. The method according to claim 55, wherein the antibody is administered every 2 weeks for the first 3 doses in 4 weeks then every 4 weeks thereafter.

58. The method according to claim 53, wherein the antibody is administered subcutaneously.

59. The method according to claim 58, wherein the antibody is administered to the patient at a dose of 200 mg a week.

60. The method according to claim 58, wherein the antibody is administered to the patient at a dose of 400 mg a week.

61. The method according to claim 58, wherein the antibody is administered at a dose of 400 mg a week for 4 weeks and then at a dose of 200 mg once weekly thereafter.

62. The method according to claim 53, wherein the antibody is administered intravenously prior to subcutaneous administration.

63. The method according to claim 62, wherein the antibody is administered intravenously at a loading dose of 10 mg/kg every 2 weeks for at least four weeks and then subcutaneously at a dose of 200 mg per week thereafter.

64. The method according to claim 52, wherein the antibody is used in combination with standard therapy.

65. The method according to claim 64, wherein the standard therapy is High Dose Corticosteroids (HDCS), Cyclophosphamide (CYC), Azathioprine (AZA), and/or Mycophenolate Mofetil (MMF).

66. The method according to claim 52, wherein the patient has biopsy-proven lupus nephritis Class III, IV, and/or V.

67. The method according to claim 52, wherein the patient has proliferative lupus nephritis.

68. The method according to claim 52, wherein the patient has membranous lupus nephritis.

69. The method according to claim 53, wherein the patient has a uPCR level of at least 4.5 g/g (uPCR≥4.5 g/g) prior to the antibody treatment.

70. The method according to claim 69, comprising administering to the patient the antibody intravenously at a dose of 10 mg/kg every 2 weeks for at least the first 12 weeks, followed by every 4 weeks thereafter.

71. The method according to claim 69, comprising administering to the patient the antibody subcutaneously at a dose of 400 mg a week for at least 12 weeks, followed by 200 mg once weekly thereafter.
